(19) United States
(12) Patent Application Publication
(10) Pub. No.: US 2008/0227790 A1
(43) Pub. Date: Sep. 18, 2008

(54) Pyridazinone Derivatives and their Use as Pde4 Inhibitors

(75) Inventors: Wiro M.P.B. Menge, Arnheim (NL); Geert Jan Sterk, Utrecht (NL)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/587,830

(22) PCT Filed: Feb. 1, 2005
(86) PCT No.: PCT/EP2005/050412

§ 371 Date: Jul. 28, 2006

(30) Foreign Application Priority Data
Feb. 4, 2004 (EP) 04002413.5

(51) Int. Cl. A61K 31/50; C07D 405/02; C07D 237/04; A61P 11/00; A61K 31/501

(52) U.S. Cl. 514/252.01; 544/238; 544/239; 514/247

(57) ABSTRACT

The compounds of a certain formula (1),

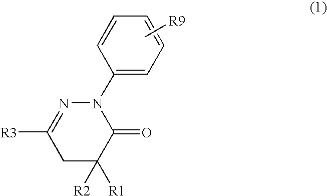

in which R1, R2, R3 and R9 have the meanings as given in the description, are novel effective PDE4 inhibitors.

### FIELD OF APPLICATION OF THE INVENTION

The invention relates to novel pyridazinone-derivatives, which are used in the pharmaceutical industry for the production of pharmaceutical compositions.

### KNOWN TECHNICAL BACKGROUND

International Patent Applications WO98/31674 (=U.S. Pat. No. 6,103,718), WO99/31071, WO99/31090, WO99/47505 (=U.S. Pat. No. 6,255,303), WO01/19818, WO01/30766, WO01/30777, WO01/94319, WO02/064584, WO02/085885 and WO02/085906 disclose phthalazinone derivatives having PDE4 inhibitory properties. In the International Patent Application WO03/032993, the European Patent Applications EP 539806, EP 618201, EP 723962, EP 738715, EP 763534 and in the German Patent Application DE19604388 arylalkyl-diazinone and thiadiazinone derivatives are described as PDE4 inhibitors. International Patent Application WO93/07146 (=U.S. Pat. No. 5,716,954) discloses benzo and pyrido pyridazinone and pyridazinthione compounds with PDE4 inhibiting activity.

In the Journal of Medicinal Chemistry, Vol. 33, No. 6, 1990, pp. 1735-1741 1,4-Bis(3-oxo-2,3-dihydropyridazin-6-yl)benzene derivatives are described as potent phosphodiesterase inhibitors and inodilators. In the Journal of Medicinal Chemistry Vol. 45 No. 12, 2002, pp. 2520-2525, 2526-2533 and in Vol. 44, No. 16, 2001, pp. 2511-2522 and pp. 2523-2535 phthalazinone derivatives are described as selective PDE4 inhibitors.

### DESCRIPTION OF THE INVENTION

It has now been found that the pyridazinone-derivatives, which are described in greater details below, have surprising and particularly advantageous properties.

The invention thus relates to compounds of formula 1

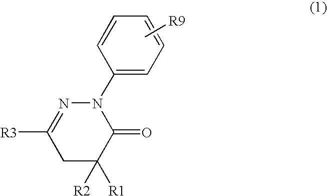

in which
R1 is 1-4C-alkyl and
R2 is 1-4C-alkyl,
R3 represents a phenyl derivative of formulae (a) or (b)

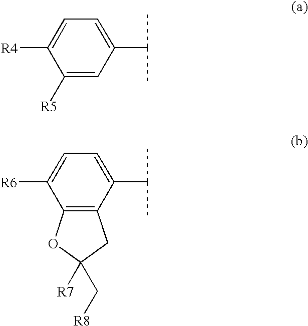

wherein
   R4 is 1-4C-alkoxy or 1-4C-alkoxy which is completely or predominantly substituted by fluorine,
   R5 is 1-8C-alkoxy, 3-7C-cycloalkoxy, 3-7C-cycloalkylmethoxy, or 1-4C-alkoxy which is completely or predominantly substituted by fluorine,
   R6 is 1-4C-alkoxy, 3-5C-cycloalkoxy, 3-5C-cycloalkylmethoxy, or 1-4C-alkoxy which is completely or predominantly substituted by fluorine,
   R7 is 1-4C-alkyl and
   R8 is hydrogen or 1-4C-alkyl,
   or wherein
   R7 and R8 together and with inclusion of the two carbon atoms, to which they are bonded, form a spiro-linked 5-, 6- or 7-membered hydrocarbon ring, optionally interrupted by an oxygen or sulphur atom,
R9 is hydroxyl, halogen, nitro, cyano, 1-4C-alkyl, trifluoromethyl, 1-4C-alkoxy, 1-4C-alkoxy which is completely or predominantly substituted by fluorine, hydroxycarbonyl, hydroxycarbonyl-1-4C-alkyl, 1-4C-alkoxycarbonyl, 1-4C-alkylcarbonyl, 1-4C-alkylcarbonylamino, 1-4C-alkylcarbonyloxy, 1-4C-alkylsulfonyl, benzyloxy, —C(O)R10, —S(O)₂—R11, —O—(CH₂)ₙ—C(O)—R12, —(CH₂)ᵣ—C(O)—R26 or —N(R29)R30,
R10 is —N(R13)R14,
R11 is —N(R22)R23,
R12 is —N(R24)R25,
R13 is hydrogen, 1-4C-alkyl, 3-7C-cycloalkyl or 3-7C-cycloalkylmethyl,
R14 is hydrogen, 1-4C-alkyl, 3-7C-cycloalkyl or 3-7C-cycloalkylmethyl,
or R13 and R14 together and with inclusion of the nitrogen atom to which they are bonded, form a 1-pyrrolidinyl-, 1-piperidinyl-, 1-hexahydroazepinyl-ring or a ring of formula (c),

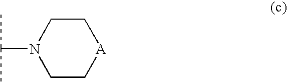

wherein
A is O, S, SO, SO₂ or NR15,
R15 is hydrogen, 1-4C-alkyl, phenyl, pyridyl, —(CH₂)ₘ—R16 or —(CH₂)ₚ—C(O)R17,
R16 is —N(R18)R19,
R17 is —N(R20)R21,
R18 is hydrogen, 1-4C-alkyl, 3-7C-cycloalkyl or 3-7C-cycloalkylmethyl,
R19 is hydrogen, 1-4C-alkyl, 3-7C-cycloalkyl or 3-7C-cycloalkylmethyl,
or R18 and R19 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl-, 4-thiomorpholinyl-, thiomorpholin-1-oxide-4-yl- or thiomorpholin-1,1-dioxide-4-yl-ring,
R20 is hydrogen, 1-4C-alkyl, 3-7C-cycloalkyl or 3-7C-cycloalkylmethyl,
R21 is hydrogen, 1-4C-alkyl, 3-7C-cycloalkyl or 3-7C-cycloalkylmethyl,
or R20 and R21 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R22 is hydrogen, 1-4C-alkyl, 3-7C-cycloalkyl or 3-7C-cycloalkylmethyl,
R23 is hydrogen, 1-4C-alkyl, 3-7C-cycloalkyl or 3-7C-cycloalkylmethyl,
or R22 and R23 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R24 is hydrogen, 1-4C-alkyl, 3-7C-cycloalkyl or 3-7C-cycloalkylmethyl,
R25 is hydrogen, 1-4C-alkyl, 3-7C-cycloalkyl or 3-7C-cycloalkylmethyl,
or R24 and R25 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R26 is —N(R27)R28,
R27 is hydrogen, 1-4C-alkyl, 3-7C-cycloalkyl or 3-7C-cycloalkylmethyl,
R28 is hydrogen, 1-4C-alkyl, 3-7C-cycloalkyl or 3-7C-cycloalkylmethyl,
or R27 and R28 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R29 is hydrogen, 1-4C-alkyl, 3-7C-cycloalkyl or 3-7C-cycloalkylmethyl,
R30 is hydrogen, 1-4C-alkyl, 3-7C-cycloalkyl or 3-7C-cycloalkylmethyl,
or R29 and R30 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
n is an integer from 1 to 2,
m is an integer from 2 to 4,
p is an integer from 1 to 4,
r is an integer from 1 to 4,
   and the salts of these compounds.

1-4C-Alkyl is a straight-chain or branched alkyl radical having 1 to 4 carbon atoms. Examples are the butyl, isobutyl, sec-butyl, tert-butyl, propyl, isopropyl, ethyl and methyl radicals.

1-4C-Alkoxy is a radical which, in addition to the oxygen atom, contains a straight-chain or branched alkyl radical having 1 to 4 carbon atoms. Alkoxy radicals having 1 to 4 carbon atoms which may be mentioned in this context are, for example, the butoxy, isobutoxy, sec-butoxy, tert-butoxy, propoxy, isopropoxy, ethoxy and methoxy radicals.

1-8C-Alkoxy is a radical which, in addition to the oxygen atom, contains a straight-chain or branched alkyl radical having 1 to 8 carbon atoms. Alkoxy radicals having 1 to 8 carbon atoms which may be mentioned in this context are, for example, the octyloxy, heptyloxy, isoheptyloxy (5-methylhexyloxy), hexyloxy, isohexyloxy (4-methylpentyloxy), neohexyloxy (3,3-dimethylbutoxy), pentyloxy, isopentyloxy (3-methylbutoxy), neopentyloxy (2,2-dimethylpropoxy), butoxy, isobutoxy, sec-butoxy, tert-butoxy, propoxy, isopropoxy, ethoxy and methoxy radicals.

1-4C-Alkoxy which is completely or predominantly substituted by fluorine is, for example, the 2,2,3,3,3-pentafluoropropoxy, the perfluoroethoxy, the 1,2,2-trifluoroethoxy and in particular the 1,1,2,2-tetrafluoroethoxy, the 2,2,2-trifluoroethoxy, the trifluoromethoxy and the difluoromethoxy radical, of which the difluoromethoxy radical is preferred. “Predominantly” in this connection means that more than half of the hydrogen atoms of the 1-4C-alkoxy group are replaced by fluorine atoms.

3-7C-Cycloalkoxy stands for cyclopropyloxy, cyclobutyloxy, cyclopentyloxy, cyclohexyloxy or cycloheptyloxy, of which cyclopropyloxy, cyclobutyloxy and cyclopentyloxy are preferred.

3-7C-Cycloalkylmethoxy stands for cyclopropylmethoxy, cyclobutylmethoxy, cyclopentylmethoxy, cyclohexylmethoxy or cycloheptylmethoxy, of which cyclopropylmethoxy, cyclobutylmethoxy and cyclopentylmethoxy are preferred.

3-5C-Cycloalkoxy stands for cyclopropyloxy, cyclobutyloxy and cyclopentyloxy.

3-5C-Cycloalkylmethoxy stands for cyclopropylmethoxy, cyclobutylmethoxy and cyclopentylmethoxy.

As spiro-linked 5,6- or 7-membered hydrocarbon rings, optionally interrupted by an oxygen or sulphur atom, may be mentioned the cyclopentane, cyclohexane, cycloheptane, tetrahydrofuran, tetrahydropyran and the tetrahydrothiophen ring.

3-7C-Cycloalkyl stands for cyclopropyl, cyclobutyl, cyclopentyl, cyclohexyl or cycloheptyl, of which cyclopropyl and cyclopentyl are preferred

3-7C-Cycloalkylmethyl stands for cyclopropylmethyl, cyclobutylmethyl, cyclopentylmethyl, cyclohexylmethyl or cycloheptylmethyl.

An hydroxycarbonyl-1-4C-alkyl radical is for example the hydroxycarbonylmethyl radical.

1-4C-Alkoxycarbonyl is a carbonyl group to which one of the abovementioned 1-4C-alkoxy radicals is bonded. Examples are the methoxycarbonyl [CH₃O—C(O)—] and the ethoxycarbonyl [CH₃CH₂O—C(O)—] radical.

1-4C-Alkylcarbonyl is a carbonyl group to which one of the abovementioned 1-4C-alkyl radicals is bonded. An example is the acetyl radical [CH₃C(O)—].

1-4C-Alkylsulfonyl is a sulfonyl group to which one of the abovementioned 1-4C-alkyl radicals is bonded. An example is the methanesulfonyl radical [CH₃S(O)₂—].

An 1-4C-Alkylcarbonylamino radical is, for example, the propionylamino [CH₇C(O)NH—] and the acetylamino radical [CH₃C(O)NH—].

1-4C-Alkylcarbonyloxy stands for a carbonyloxy group to which one of the abovementioned 1-4C-alkyl radicals is bonded. An example is the acetoxy radical [CH₃C(O)—O—].

Suitable salts for compounds of formula 1 are—depending on substitution—all acid addition salts or all salts with bases. Particular mention may be made of the pharmacologically tolerable inorganic and organic acids and bases customarily used in pharmacy. Those suitable are, on the one hand, water-soluble and water-insoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in salt preparation—depending on whether a mono- or polybasic acid is concerned and depending on which salt is desired—in an equimolar quantitative ratio or one differing therefrom.

On the other hand, salts with bases are—depending on substitution—also suitable. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

Pharmacologically intolerable salts, which can be obtained, for example, as process products during the preparation of the compounds according to the invention on an industrial scale, are converted into pharmacologically tolerable salts by processes known to the person skilled in the art.

According to expert's knowledge the compounds of the invention as well as their salts may contain, e.g. when isolated in crystalline form, varying amounts of solvents. Included within the scope of the invention are therefore all solvates and in particular all hydrates of the compounds of formula 1 as well as all solvates and in particular all hydrates of the salts of the compounds of formula 1.

An embodiment (embodiment A) of the invention are those compounds of formula 1 in which
R1 is 1-4C-alkyl,
R2 is 1-4C-alkyl,
R3 represents a phenyl derivative of formulae (a) or (b)

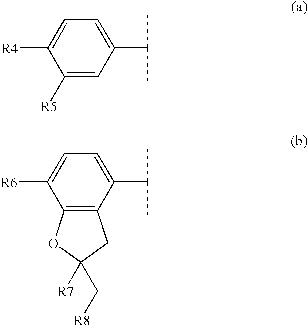

wherein
   R4 is 1-2C-alkoxy or 1-2C-alkoxy which is completely or predominantly substituted by fluorine,
   R5 is 1-2C-alkoxy or 1-2C-alkoxy which is completely or predominantly substituted by fluorine,
   R6 is 1-2C-alkoxy or 1-2C-alkoxy which is completely or predominantly substituted by fluorine,
   R7 is methyl and
   R8 is hydrogen,
   or wherein
   R7 and R8 together and with inclusion of the two carbon atoms, to which they are bonded, form a spiro-linked cyclopentane, cyclohexane, tetrahydrofuran or tetrahydropyran ring,
R9 is hydroxyl, halogen, hydroxycarbonyl, hydroxycarbonyl-1-4C-alkyl, benzyloxy, —C(O)R10, —S(O)₂—R11, —O—(CH₁)ₙ—C(O)—R12, —(CH₂)ᵣ—C(O)—R26 or —N(R29)R30,
R10 is —N(R13)R14,
R11 is —N(R22)R23,
R12 is —N(R24)R25,
R13 is hydrogen or 1-4C-alkyl,
R14 is hydrogen or 1-4C-alkyl,
or R13 and R14 together and with inclusion of the nitrogen atom to which they are bonded, form a 1-pyrrolidinyl-, 1-piperidinyl-, 1-hexahydroazepinyl-ring or a ring of formula (c),

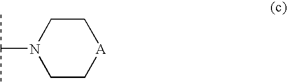

wherein
A is O, S, SO, SO₂ or NR15,
R15 is hydrogen, 1-4C-alkyl, phenyl, pyridyl, —(CH₂)ₘ—R16 or —(CH₂)ₚ—C(O)R17,
R16 is —N(R18)R19,
R17 is —N(R20)R21,
R18 is hydrogen or 1-4C-alkyl,
R19 is hydrogen or 1-4C-alkyl,
or R18 and R19 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl-, 4-thiomorpholinyl-, thiomorpholin-1-oxide-4-yl- or thiomorpholin-1,1-dioxide-4-yl-ring,
R20 is hydrogen or 1-4C-alkyl,
R21 is hydrogen or 1-4C-alkyl,
or R20 and R21 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R22 is hydrogen or 1-4C-alkyl,
R23 is hydrogen or 1-4C-alkyl,
or R22 and R23 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R24 is hydrogen or 1-4C-alkyl,
R25 is hydrogen or 1-4C-alkyl,
or R24 and R25 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R26 is —N(R27)R28,
R27 is hydrogen or 1-4C-alkyl,
R28 is hydrogen or 1-4C-alkyl,
or R27 and R28 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R29 is hydrogen or 1-4C-alkyl,
R30 is hydrogen or 1-4C-alkyl,
or R29 and R30 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
n is an integer from 1 to 2,
m is an integer from 2 to 4,
p is an integer from 1 to 4,
r is an integer from 1 to 4,
   and the salts of these compounds.

Subgroup 1 of embodiment A to be emphasized are those compounds of formula 1 in which
R1 is methyl or ethyl,
R2 is methyl or ethyl,
R3 represents a phenyl derivative of formulae (a) or (b)

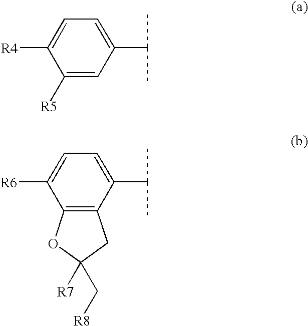

wherein
R4 is methoxy, ethoxy or difluoromethoxy,
R5 is methoxy, ethoxy or difluoromethoxy,
R6 is methoxy, ethoxy or difluoromethoxy,
R7 is methyl and
R8 is hydrogen,
or wherein
R7 and R8 together and with inclusion of the two carbon atoms, to which they are bonded, form a spiro-linked cyclopentane, cyclohexane, tetrahydrofuran or tetrahydropyran ring,
R9 is hydroxyl, halogen, hydroxycarbonyl, hydroxycarbonylmethyl or benzyloxy, and the salts of these compounds.

Subgroup 2 of embodiment A to be emphasized are those compounds of formula 1 in which
R1 is methyl or ethyl,
R2 is methyl or ethyl,
R3 represents a phenyl derivative of formulae (a) or (b)

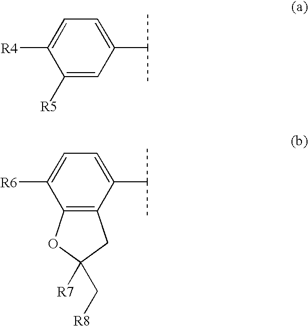

wherein
   R4 is methoxy, ethoxy or difluoromethoxy,
   R5 is methoxy, ethoxy or difluoromethoxy,
   R6 is methoxy, ethoxy or difluoromethoxy,
   R7 is methyl and
   R8 is hydrogen,
   or wherein
   R7 and R8 together and with inclusion of the two carbon atoms, to which they are bonded, form a spiro-linked cyclopentane, cyclohexane, tetrahydrofuran or tetrahydropyran ring,
R9 is —C(O)R10,
R10 is —N(R13)R14,
R13 is hydrogen or 1-4C-alkyl,
R14 is hydrogen or 1-4C-alkyl,
or R13 and R14 together and with inclusion of the nitrogen atom to which they are bonded, form a 1-pyrrolidinyl-, a 1-piperidinyl-ring or a ring of formula (c),

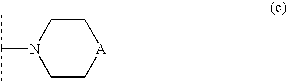

wherein
A is O, S, SO₂ or NR15,
R15 is 1-4C-alkyl, phenyl, pyridyl, —(CH₂)ₘR16 or —(CH₂)ₚ—C(O)R17,
R16 is —N(R18)R19,
R17 is —N(R20)R21,
R18 is hydrogen or 1-4C-alkyl,
R19 is hydrogen or 1-4C-alkyl,
or R18 and R19 together and with inclusion of the nitrogen atom to which they are bonded, form a 1-pyrrolidinyl-, 1-piperidinyl-, 1-methyl-piperazin-4-yl- or a 4-morpholinyl-ring,
R20 is hydrogen or 1-4C-alkyl,
R21 is hydrogen or 1-4C-alkyl,
or R20 and R21 together and with inclusion of the nitrogen atom to which they are bonded, form a 1-pyrrolidinyl-, 1-piperidinyl-, 1-methyl-piperazin-4-yl- or a 4-morpholinyl-ring,
m is 2,
p is 1,
   and the salts of these compounds.

Subgroup 3 of embodiment A to be emphasized are those compounds of formula 1 in which
R1 is methyl or ethyl,
R2 is methyl or ethyl,
R3 represents a phenyl derivative of formulae (a) or (b)

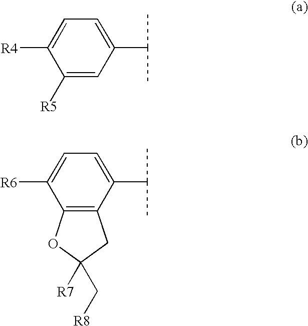

wherein
   R4 is methoxy, ethoxy or difluoromethoxy,
   R5 is methoxy, ethoxy or difluoromethoxy,
   R6 is methoxy, ethoxy or difluoromethoxy,
   R7 is methyl and
   R8 is hydrogen,
   or wherein
   R7 and R8 together and with inclusion of the two carbon atoms, to which they are bonded, form a spiro-linked cyclopentane, cyclohexane, tetrahydrofuran or tetrahydropyran ring,
R9 is —S(O)₂—R11,
R11 is —N(R22)R23,
R22 is hydrogen or 1-4C-alkyl,
R23 is hydrogen or 1-4C-alkyl,
or R22 and R23 together and with inclusion of the nitrogen atom to which they are bonded, form a 1-pyrrolidinyl-, 1-piperidinyl-, 1-methyl-piperazin-4-yl- or a 4-morpholinyl-ring,
   and the salts of these compounds.

Subgroup 4 of embodiment A to be emphasized are compounds of formula 1 in which
R1 is methyl or ethyl,
R2 is methyl or ethyl,
R3 represents a phenyl derivative of formulae (a) or (b)

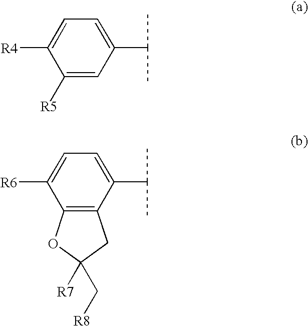

wherein
   R4 is methoxy, ethoxy or difluoromethoxy,
   R5 is methoxy, ethoxy or difluoromethoxy,
   R6 is methoxy, ethoxy or difluoromethoxy,
   R7 is methyl and
   R8 is hydrogen,
   or wherein
   R7 and R8 together and with inclusion of the two carbon atoms, to which they are bonded, form a spiro-linked cyclopentane, cyclohexane, tetrahydrofuran or tetrahydropyran ring,
R9 is —O—(CH₂)ₙ—C(O)—R12,
R12 is —N(R24)R25,
R24 is hydrogen or 1-4C-alkyl,
R25 is hydrogen or 1-4C-alkyl,
or R24 and R25 together and with inclusion of the nitrogen atom to which they are bonded, form a 1-pyrrolidinyl-, 1-piperidinyl-, 1-methyl-piperazin-4-yl- or a 4-morpholinyl-ring,
n is 1,
   and the salts of these compounds.

Subgroup 5 of embodiment A to be emphasized are compounds of formula 1 in which
R1 is methyl or ethyl,
R2 is methyl or ethyl,
R3 represents a phenyl derivative of formulae (a) or (b)

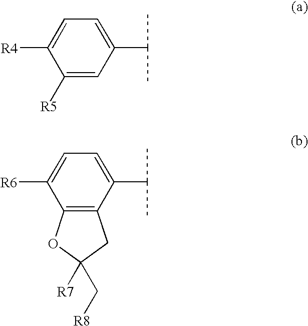

wherein
   R4 is methoxy, ethoxy or difluoromethoxy,
   R5 is methoxy, ethoxy or difluoromethoxy,
   R6 is methoxy, ethoxy or difluoromethoxy,
   R7 is methyl and
   R8 is hydrogen,
   or wherein
   R7 and R8 together and with inclusion of the two carbon atoms, to which they are bonded, form a spiro-linked cyclopentane, cyclohexane, tetrahydrofuran or tetrahydropyran ring,
R9 is —(CH₂)ᵣ—C(O)—R26,
R26 is —N(R27)R28,
R27 is hydrogen or 1-4C-alkyl,
R28 is hydrogen or 1-4C-alkyl,
or R27 and R28 together and with inclusion of the nitrogen atom to which they are bonded, form a 1-pyrrolidinyl-, 1-piperidinyl-, 1-methyl-piperazin-4-yl- or a 4-morpholinyl-ring,
r is 1,
   and the salts of these compounds.

Subgroup 6 of embodiment A to be emphasized are compounds of formula 1 in which
R1 is methyl or ethyl,
R2 is methyl or ethyl,
R3 represents a phenyl derivative of formulae (a) or (b)

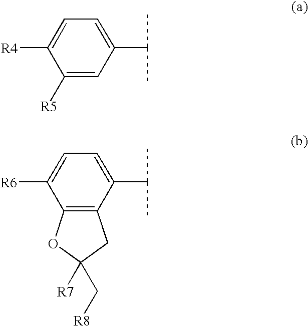

wherein
   R4 is methoxy, ethoxy or difluoromethoxy,
   R5 is methoxy, ethoxy or difluoromethoxy,
   R6 is methoxy, ethoxy or difluoromethoxy,
   R7 is methyl and
   R8 is hydrogen,
   or wherein
   R7 and R8 together and with inclusion of the two carbon atoms, to which they are bonded, form a spiro-linked cyclopentane, cyclohexane, tetrahydrofuran or tetrahydropyran ring,
R9 is —N(R29)R30,
R29 and R30 together and with inclusion of the nitrogen atom to which they are bonded, form a 1-pyrrolidinyl-, 1-piperidinyl-, 1-methyl-piperazin-4-yl- or a 4-morpholinyl-ring,
   and the salts of these compounds.

Subgroup 1 of embodiment A to be particularly emphasized are those compounds of formula 1 in which
R1 is methyl,
R2 is methyl,
R3 represents a phenyl derivative of formulae (a) or (b)

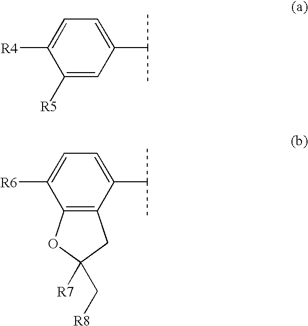

wherein
   R4 is methoxy or ethoxy,
   R5 is methoxy or ethoxy,
   R6 is methoxy,
   R7 is methyl and
   R8 is hydrogen,
R9 is hydroxyl, bromine, hydroxycarbonyl, hydroxycarbonylmethyl or benzyloxy,
   and the salts of these compounds.

Subgroup 2 of embodiment A to be particularly emphasized are those compounds of formula 1 in which
R1 is methyl,
R2 is methyl,
R3 represents a phenyl derivative of formulae (a) or (b)

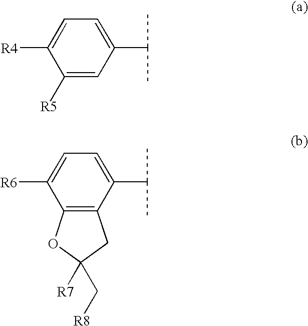

wherein
   R4 is methoxy or ethoxy,
   R5 is methoxy or ethoxy,
   R6 is methoxy,
   R7 is methyl and
   R8 is hydrogen,
R9 is —C(O)R10,
R10 is —N(R13)R14,
R13 and R14 together and with inclusion of the nitrogen atom to which they are bonded, form a ring of formula (c),

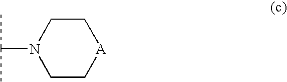

wherein
A is O or NR15,
R15 is pyridyl, —(CH₂)ₘ—R16 or —(CH₂)ₚ—C(O)R17,
R16 is —N(R18)R19,
R17 is —N(R20)R21,
R18 and R19 together and with inclusion of the nitrogen atom to which they are bonded, form a 4-morpholinyl-ring,
R20 and R21 together and with inclusion of the nitrogen atom to which they are bonded, form a 1-pyrrolidinyl-ring,
m is 2,
p is 1,
   and the salts of these compounds.

Subgroup 3 of embodiment A to be particularly emphasized are those compounds of formula 1 in which
R1 is methyl,
R2 is methyl,
R3 represents a phenyl derivative of formulae (a) or (b)

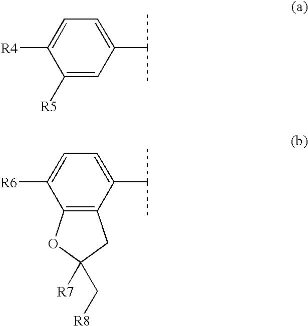

wherein
   R4 is methoxy or ethoxy,
   R5 is methoxy or ethoxy,
   R6 is methoxy,
   R7 is methyl and
   R8 is hydrogen,
R9 is —S(O)₂—R11,
R11 is —N(R22)R23,
R22 and R23 together and with inclusion of the nitrogen atom to which they are bonded, form a 4-morpholinyl-ring,
   and the salts of these compounds.

Subgroup 4 of embodiment A to be particularly emphasized are those compounds of formula 1 in which
R1 is methyl,
R2 is methyl,
R3 represents a phenyl derivative of formulae (a) or (b)

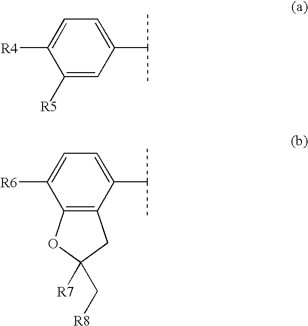

wherein
   R4 is methoxy or ethoxy,
   R5 is methoxy or ethoxy,
   R6 is methoxy,
   R7 is methyl and
   R8 is hydrogen,
R9 is —O—(CH₂)ₙ—C(O)—R12,
R12 is —N(R24)R25,
R24 is hydrogen,
R25 is hydrogen,
or R24 and R25 together and with inclusion of the nitrogen atom to which they are bonded, form a 1-pyrrolidinyl-, 1-methyl-piperazin-4-yl- or a 4-morpholinyl-ring,
n is 1,
   and the salts of these compounds.

Subgroup 5 of embodiment A to be particularly emphasized are those compounds of formula 1 in which
R1 is methyl,
R2 is methyl,
R3 represents a phenyl derivative of formulae (a) or (b)

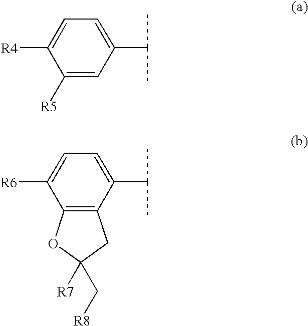

wherein
R4 is methoxy or ethoxy,
R5 is methoxy or ethoxy,
R6 is methoxy,
R7 is methyl and
R8 is hydrogen,
R9 is —(CH₂)ᵣ—C(O)—R26,
R26 is —N(R27)R28,
R27 and R28 together and with inclusion of the nitrogen atom to which they are bonded, form a 4-morpholinyl-ring,
r is 1,
   and the salts of these compounds.

Subgroup 6 of embodiment A to be particularly emphasized are those compounds of formula 1 in which
R1 is methyl,
R2 is methyl,
R3 represents a phenyl derivative of formulae (a) or (b)

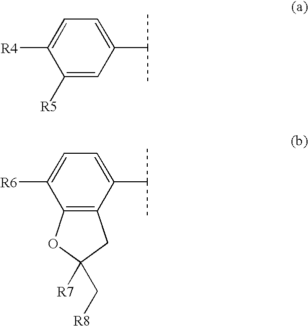

wherein
   R4 is methoxy or ethoxy,
   R5 is methoxy or ethoxy,
   R6 is methoxy,
   R7 is methyl and
   R8 is hydrogen,
R9 is —N(R29)R30,
R29 and R30 together and with inclusion of the nitrogen atom to which they are bonded, form a 4-morpholinyl-ring,
   and the salts of these compounds.

Preferred compounds of subgroup 2 of embodiment A are those compounds of formula 1 in which
R1 is methyl,
R2 is methyl,
R3 represents a phenyl derivative of formulae (a) or (b)

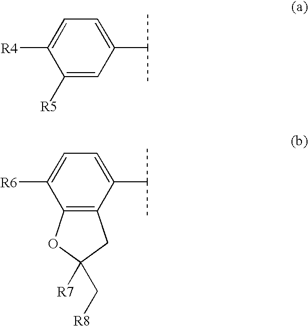

wherein
   R4 is methoxy or ethoxy,
   R5 is methoxy or ethoxy,
   R6 is methoxy,
   R7 is methyl and
   R8 is hydrogen,
R9 is —C(O)R10,
R10 is —N(R13)R14,
R13 and R14 together and with inclusion of the nitrogen atom to which they are bonded, form a ring of formula (c),

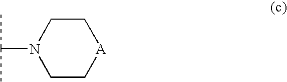

wherein
A is O,
   and the salts of these compounds.

Another embodiment (embodiment B) of the invention are those compounds of formula 1 in which
R1 is 1-4C-alkyl and
R2 is 1-4C-alkyl,
R3 represents a phenyl derivative of formulae (a) or (b)

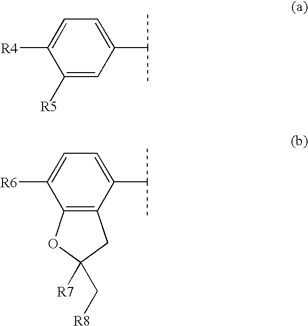

wherein
   R4 is 1-4C-alkoxy or 1-4C-alkoxy which is completely or predominantly substituted by fluorine,
   R5 is 1-8C-alkoxy, 3-7C-cycloalkoxy, 3-7C-cycloalkylmethoxy, or 1-4C-alkoxy which is completely or predominantly substituted by fluorine,
   R6 is 1-4C-alkoxy, 3-5C-cycloalkoxy, 3-5C-cycloalkylmethoxy, or 1-4C-alkoxy which is completely or predominantly substituted by fluorine,
   R7 is 1-4C-alkyl and
   R8 is hydrogen or 1-4C-alkyl,
   or wherein
   R7 and R8 together and with inclusion of the two carbon atoms, to which they are bonded, form a spiro-linked 5-, 6- or 7-membered hydrocarbon ring, optionally interrupted by an oxygen or sulphur atom,
R9 is hydroxyl, halogen, nitro, cyano, hydroxycarbonyl, 1-4C-alkyl, trifluoromethyl, 1-4C-alkoxy, 1-4C-alkoxy which is completely or predominantly substituted by fluorine, amino, mono- or di-1-4C-alkylamino, 1-4C-alkylcarbonylamino, 1-4C-alkylcarbonyloxy, benzyloxy, —C(O)R10, —S(O)₂—R11 or —O—(CH₂)ₙ—C(O)—R12,
R10 is 1-4C-alkyl, 1-4C-alkoxy or —N(R13)R14,
R11 is 1-4C-alkyl or —N(R22)R23,
R12 is —N(R24)R25,
R13 is hydrogen, 1-4C-alkyl, 3-7C-cycloalkyl or 3-7C-cycloalkylmethyl,
R14 is hydrogen, 1-4C-alkyl, 3-7C-cycloalkyl or 3-7C-cycloalkylmethyl,
or R13 and R14 together and with inclusion of the nitrogen atom to which they are bonded, form a 1-pyrrolidinyl-, 1-piperidinyl-, 1-hexahydroazepinyl-ring or a ring of formula (c),

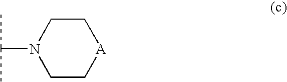

wherein
A is O, S, SO, SO₂ or NR15,
R15 is hydrogen, 1-4C-alkyl, phenyl, pyridyl, —(CH₂)ₘ—R16 or —(CH₂)ₚ—C(O)R17,
R16 is —N(R18)R19,
R17 is —N(R20)R21,
R18 is hydrogen, 1-4C-alkyl, 3-7C-cycloalkyl or 3-7C-cycloalkylmethyl,
R19 is hydrogen, 1-4C-alkyl, 3-7C-cycloalkyl or 3-7C-cycloalkylmethyl,
or R18 and R19 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl-, 4-thiomorpholinyl-, thiomorpholin-1-oxide-4-yl- or thiomorpholin-1,1-dioxide-4-yl-ring,
R20 is hydrogen, 1-4C-alkyl, 3-7C-cycloalkyl or 3-7C-cycloalkylmethyl,
R21 is hydrogen, 1-4C-alkyl, 3-7C-cycloalkyl or 3-7C-cycloalkylmethyl,
or R20 and R21 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R22 is hydrogen, 1-4C-alkyl, 3-7C-cycloalkyl or 3-7C-cycloalkylmethyl,
R23 is hydrogen, 1-4C-alkyl, 3-7C-cycloalkyl or 3-7C-cycloalkylmethyl,
or R22 and R23 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R24 is hydrogen, 1-4C-alkyl, 3-7C-cycloalkyl or 3-7C-cycloalkylmethyl,
R25 is hydrogen, 1-4C-alkyl, 3-7C-cycloalkyl or 3-7C-cycloalkylmethyl,
or R24 and R25 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
n is an integer from 1 to 2,
m is an integer from 2 to 4,
p is an integer from 1 to 4,
   and the salts of these compounds.

Compounds of formula 1 of embodiment B to be emphasized are those in which
R1 is 1-4C-alkyl,
R2 is 1-4C-alkyl,
R3 represents a phenyl derivative of formulae (a) or (b)

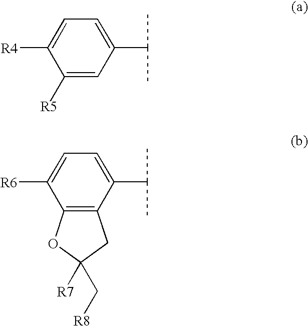

wherein
   R4 is 1-2C-alkoxy or 1-2C-alkoxy which is completely or predominantly substituted by fluorine,
   R5 is 1-4C-alkoxy,
   R6 is 1-2C-alkoxy or 1-2C-alkoxy which is completely or predominantly substituted by fluorine,
   R7 is methyl and
   R8 is hydrogen,
   or wherein
   R7 and R8 together and with inclusion of the two carbon atoms, to which they are bonded, form a spiro-linked cyclopentane, cyclohexane, tetrahydrofurane or tetrahydropyran ring,
R9 is hydroxyl, hydroxycarbonyl, benzyloxy, —C(O)R1 or —O—(CH₂)ᵣ—C(O)—R12,
R10 is —N(R13)R14,
R12 is —N(R24)R25,
R13 is hydrogen or 1-4C-alkyl,
R14 is hydrogen or 1-4C-alkyl,
or R13 and R14 together and with inclusion of the nitrogen atom to which they are bonded, form a 1-pyrrolidinyl-, 1-piperidinyl-, 1-hexahydroazepinyl-ring or a ring of formula (c),

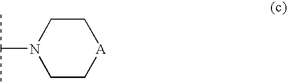

wherein
A is O, S, SO, SO₂ or NR15,
R15 is hydrogen, 1-4C-alkyl, phenyl, pyridyl, —(CH₂)ₘ—R16 or —(CH₂)ₚ—C(O)R17,
R16 is —N(R18)R19,
R17 is —N(R20)R21,
R18 is hydrogen or 1-4C-alkyl,
R19 is hydrogen or 1-4C-alkyl,
or R18 and R19 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl-, 4-thiomorpholinyl-, thiomorpholin-1-oxide-4-yl- or thiomorpholin-1,1-dioxide-4-yl-ring,
R20 is hydrogen or 1-4C-alkyl,
R21 is hydrogen or 1-4C-alkyl,
or R20 and R21 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R24 is hydrogen or 1-4C-alkyl,
R25 is hydrogen or 1-4C-alkyl,
or R24 and R25 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
n is an integer from 1 to 2,
m is an integer from 2 to 4,
p is an integer from 1 to 4,
   and the salts of these compounds.

Preferred compounds of formula 1 of embodiment B are those, in which
R1 is methyl,
R2 is methyl,
R3 represents a phenyl derivative of formula (a)

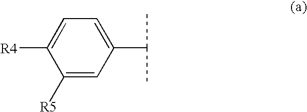

wherein
   R4 is methoxy or ethoxy,
   R5 is methoxy or ethoxy,
R9 is hydroxyl, hydroxycarbonyl, benzyloxy, —C(O)R10 or —O—(CH₂)ₙ—C(O)—R12,
R10 is —N(R13)R14,
R12 is —N(R24)R25,
R13 and R14 together and with inclusion of the nitrogen atom to which they are bonded, form a ring of formula (c),

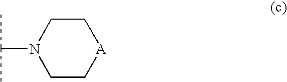

wherein
A is O or NR15,
R15 is pyrid-4-yl, —(CH₂)ₘ—R16 or —(CH₂)ₚ—C(O)R17,
R16 is 4-morpholinyl,
R17 is 1-pyrrolidinyl,
R24 is hydrogen,
R25 is hydrogen,
or R24 and R25 together and with inclusion of the nitrogen atom to which they are bonded, form a 1-methyl-piperazin-4-yl- or 4-morpholinyl-ring,
n is 1,
m is 2,
p is 1,
   and the salts of these compounds.

A special embodiment of the compounds of the present invention includes those compounds of formula 1 in which R3 represents a phenyl derivative of formula (a).

Another special embodiment of the compounds of the present invention includes those compounds of formula 1 in which R3 represents a phenyl derivative of formula (a) and R4 and R5 have the meaning methoxy.

Still another special embodiment of the compounds of the present invention includes those compounds of formula 1 in which R1 is methyl, R2 is methyl, R3 represents a phenyl derivative of formula (a) and R4 and R5 have the meaning methoxy.

A further special embodiment of the compounds of the present invention includes those compounds of formula 1 in which R3 represents a phenyl derivative of formula (b).

Still a further special embodiment of the compounds of the present invention includes those compounds of formula 1 in which R3 represents a phenyl derivative of formula (b) and R6 is methoxy, R7 is methyl and R8 is hydrogen.

Another special embodiment of the compounds of the present invention includes those compounds of formula 1 in which R1 is methyl, R2 is methyl, R3 represents a phenyl derivative of formula (b), R6 is methoxy, R7 is methyl and R8 is hydrogen.

The compounds of formula 1 are chiral compounds, if the meanings of R1 and R2 are not identical. In case R3 represents a phenyl derivative of formula (b) there is one further chiral center in the dihydrofuran-ring, if the substituents —R7 and —CH₂R8 are not identical. However, preferred are in this connection those compounds, in which the substituents —R7 and —CH₂R8 are identical or together and with inclusion of the two carbon atoms to which they are bonded form a spiro-connected 5-, 6- or 7-membered hydrocarbon ring.

The invention includes all conceivable pure diastereomers and pure enantiomers of the compounds of formula 1, as well as all mixtures thereof independent from the ratio, including the racemates.

The compounds of formula 1 according to the invention can be prepared, for example, as described in Reaction schemes 1 and 2.

In reaction scheme 1 the preparation of compounds of formula 1, in which R1, R2 and R3 have the above-mentioned meanings and R9 is hydroxycarbonyl or —C(O)R10 is described.

In reaction scheme 2 the preparation of compounds of formula 1, in which R1, R2 and R3 have the above-mentioned meanings and R9 is hydroxyl, benzyloxy or —O—(CH₂)ₙ—C(O)—R12 is described.

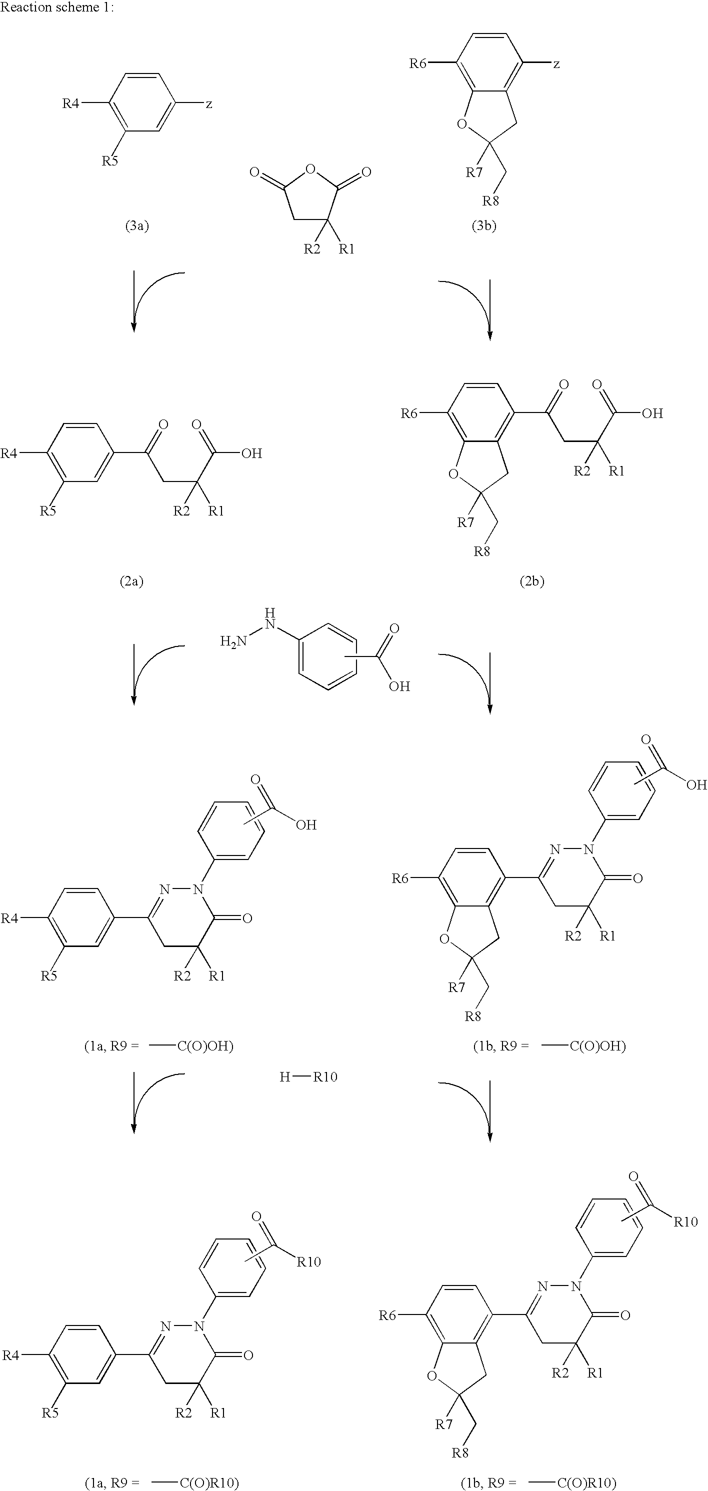

In reaction scheme 1, the keto acids of formula 2a, in which R1, R2, R4 and R5 have the above-mentioned meanings, can, for example, be prepared from compounds of formula 3a, in which R4 and R5 have the above-mentioned meanings and Z represents hydrogen (H) by a Friedel-Crafts acylation with 3,3-di-(1-4C-alkyl)-dihydro-furan-2,5-dione (for example 3,3-di-methyl-dihydro-furan-2,5-dione or 3,3-di-ethyl-dihydro-furan-2,5-dione). The Friedel-Crafts acylation is carried out in a manner, which is known to the person skilled in the art (for example as described in M. Yamaguchi et al., J Med Chem 36: 4052-4060, 1993) in presence of a suitable catalyst, such as for example, AlCl₃, ZnCl₂, FeCl₃ or iodine, in an appropriate inert solvent, such as methylene chloride or nitrobenzene or another inert solvent such as diethyl ether, preferably at raised temperature, especially at the boiling point of the solvent being used.

Alternatively, the compounds of formula 2a, in which R1, R2, R4 and R5 have the above-mentioned meanings, can be prepared from compounds of the formula 3a, in which R4 and R5 have the above-mentioned meanings and Z represents a halogen atom through reaction with 3,3-di-(1-4C-alkyl)-dihydro-furan-2,5-dione.

The reaction is carried out in a manner, which is known by a person skilled in the art, for example
a) by activating compounds of formula 3a, in which R4, R5 and Z have the above-mentioned meanings, by a lithium/halogen exchange reaction at low temperatures (preferably at −60 to −100° C.) in an appropriate inert solvent such as tetrahydrofuran or diethylether, preferably under an atmosphere of inert gas, followed by reaction of the lithiated compounds with 3,3-di-(1-4C-alkyl)-dihydro-furan-2,5-dione, or
b) by converting compounds of formula 3a, in which R4, R5 and Z have the above-mentioned meanings, in a suitable inert solvent such as, for example, tetrahydrofuran or diethyl ether into the corresponding Grignard compounds of formulae 3a and 3b, in which Z represents MgCl, MgBr or MgI followed by reaction of the Grignard compounds with 3,3-di-(1-4C-alkyl)-dihydro-furan-2,5-dione.

Compounds of formula 2b, in which R1, R2, R6, R7 and R8 have the above-mentioned meanings can be prepared analogously to the compounds of formula 2a using the synthesis procedures described above under a) or b).

Compounds of formula 3a, in which R4 and R5 have the above-mentioned meanings and Z represents a hydrogen (H) or halogen atom, are known or can be prepared as described in WO98/31674.

Compounds of formula 3b, in which R6, R7 and R8 have the above-mentioned meanings and Z represents a halogen atom, are known or can be prepared as described in WO99/31090.

The keto acids of formulae 2a and 2b are converted to compounds of formula 1a and 1b, in which R1, R2, R4, R5, R6, R7 and R8 have the above-mentioned meanings and R9 represents hydroxycarbonyl by a reaction with a hydrazinobenzoic acid derivative.

The conversion of the keto acids of formulae 2a and 2b or one of their reactive derivatives with hydrazinobenzoic acid is advantageously carried out with 1 to 1.5 equivalents of the hydrazinobenzoic acid. Preferably, pyridine is used as inert solvent. Other inert solvents which can be used are alcohols such as methanol, ethanol, isopropanol, n-butanol, isoamylalcohol, glycols and their ethers such as ethylene glycol, diethylene glycol, ethylene glycol monomethyl or monoethyl ether, acids such as formic acid, acetic or propionic acid, suitable mixtures of the above-mentioned solvents, as well as mixtures with water, for example aqueous ethanol, further ethers, especially water soluble ethers such as tetrahydrofuran, dioxane or ethylene glycol dimethylether; further toluene or benzene, especially when the method of azeotropic destillation is used to remove the reaction water.

The reaction temperatures are suitably between 0 and 200° C., preferably between 20 and 100° C.; the reaction times are preferably between 1 and 48 hours.

Suitable reactive derivatives of the keto acids of formulae 2a and 2b which may be mentioned in this context are, for example, esters, especially methyl and ethyl esters, nitrils and acid halides, such as acid chlorides or acid bromides. They can be prepared by methods which are known by the person skilled in the art.

Finally, the compounds of formulae 1a and 1b, in which R1, R2, R4, R5, R6, R7 and R8 have the above-mentioned meanings and R9 has the meaning hydroxycarbonyl can be converted into further compounds of formula 1 by reaction with a compound of formula R10-H, in which R10 has the above-mentioned meanings.

All known classical methods for the formation of an amide can be used for this conversion.

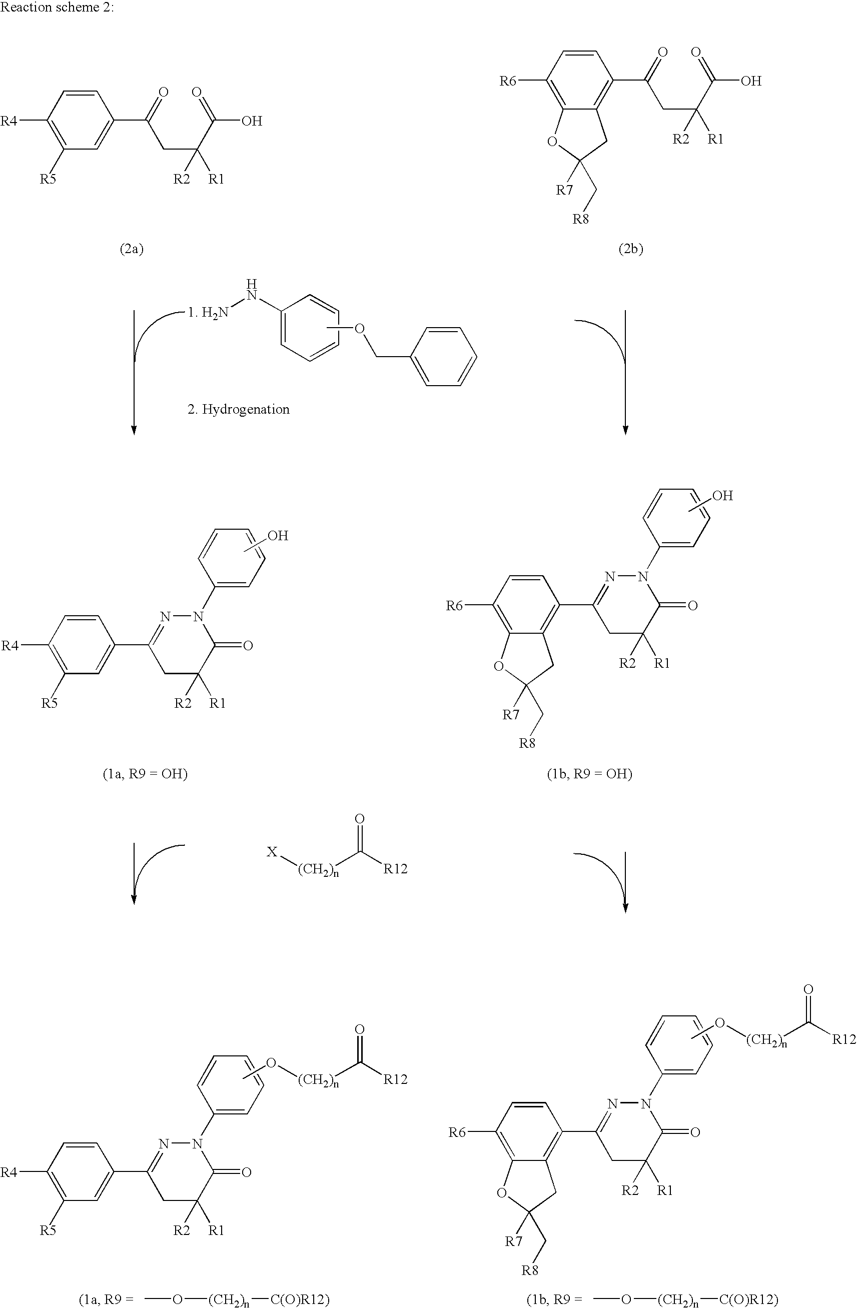

In reaction scheme 2 the keto acids of formulae 2a and 2b, in which R1, R2, R4, R5, R6, R7 and R8 have the above-mentioned meanings, are reacted with benzyloxyphenylhydrazine to give the corresponding compounds of formulae 1a and 1b, in which R9 has the meaning benzyloxy.

In a hydrogenation step the benzyl is removed to yield compounds of formula 1a and 1b, in which R1, R2, R4, R5, R6, R7 and R8 have the above-mentioned meanings and R9 has the meaning hydroxyl.

Finally, the compounds of formulae 1a and 1b, in which R1, R2, R4, R5, R6, R7 and R8 have the above-mentioned meanings and R9 has the meaning hydroxyl can be converted into further compounds of formulae 1a and 1b by reaction with compounds of formula R12-C(O)—(CH₂)ₙ—X, in which R12 and n have the above-mentioned meanings and X is a suitable leaving group, for example a halogen atom, preferably a chlorine atom.

All classical methods, which have been described for the alkylation of a phenol, such as for example Williamson's ether synthesis or the Mitsunobu reaction may be used in this reaction.

Compounds of formula 1, in which R1, R2, R3, R4, R5, R6, R7 and R8 have the above-mentioned meanings and R9 is —S(O)₂—R11 can be prepared analogously as described in reaction scheme 1 using instead of hydrazinobenzoic acid hydrazinobenzenesulfonic acid and instead of compounds of formula R10-H compounds of formula R11-H.

Compounds of formula 1, in which R1, R2, R3, R4, R5, R6, R7 and R8 have the above-mentioned meanings and R9 is halogen, nitro, cyano, 1-4C-alkyl, trifluoromethyl, 1-4C-alkoxy, 1-4C-alkoxy which is completely or predominantly substituted by fluorine, hydroxycarbonyl-1-4C-alkyl, 1-4C-alkoxycarbonyl, 1-4C-alkylcarbonyl 1-4C-alkylcarbonylamino, 1-4C-alkylcarbonyloxy, 1-4C-alkylsulfonyl can be prepared analogously as described in the first reaction step of reaction scheme 2 using instead of benzyloxyphenylhydrazine suitable substituted phenylhydrazine derivatives.

Compounds of formula 1, in which R1, R2, R3, R4, R5, R6, R7 and R8 have the above-mentioned meanings and R9 is —(CH₂)ᵣ—C(O)—R26, wherein r and R26 have the above-mentioned meanings can be prepared analogously as described in reaction scheme 1 using instead of hydrazinebenzoic acid hydrazinophenyl-acetic acid (hydrazinophenyl-propionic acid or hydrazinophenyl-butyric acid) and instead of compounds of formula R10-H compounds of formula R26-H.

Compounds of formula 1, in which R1, R2, R3, R4, R5, R6, R7 and R8 have the above-mentioned meanings and R9 is —N(R29)R30 can be prepared, for example, as described in the section “Examples”.

Suitably, the conversions are carried out analogous to methods, which are familiar per se to the person skilled in the art, for example, in the manner which is described in the following examples.

The substances according to the invention are isolated and purified in a manner known per se, e.g. by distilling off the solvent in vacuo and recrystallising the residue obtained from a suitable solvent or subjecting it to one of the customary purification methods, such as column chromatography on a suitable support material.

Salts are obtained by dissolving the free compound in a suitable solvent (for example a ketone like acetone, methylethylketone, or methylisobutylketone, an ether, like diethyl ether, tetrahydrofuran or dioxane, a chlorinated hydrocarbon, such as methylene chloride or chloroform, or a low molecular weight aliphatic alcohol, such as ethanol, isopropanol) which contains the desired acid, or to which the desired acid is then added. The salts are obtained by filtering, reprecipitating, precipitating with a non-solvent for the addition salt or by evaporating the solvent. Salts obtained can be converted by basification into the free compounds which, in turn, can be converted into salts. In this manner, pharmacologically non-tolerable salts can be converted into pharmacologically tolerable salts.

The following examples illustrate the invention in greater detail, without restricting it. As well, further compounds of formula 1, of which the preparation is explicitly not described, can be prepared in an analogous way or in a way which is known by a person skilled in the art using customary preparation methods.

The compounds, which are mentioned in the examples as well as their salts are preferred compounds of the invention. In the examples, RT stands for room temperature, h for hour(s), min for minute(s) and M.p. for melting point.

### EXAMPLES

### Final Products

### 1. 4-(3-{3,4-dimethoxyphenyl}-5,5-dimethyl-6-oxo-5,6-dihydro-4H-pyridazin-1-yl)-benzoic acid

A mixture of 20 mmol of intermediate A1, 25 mmol of 4-hydrazinobenzoic acid and 5 g pyridine hydrochloride in 150 ml of pyridine is heated under reflux for 72 h. The solvent is evaporated and the residue is partitioned between 1N hydrochloric acid and dichloromethane. The dichloromethane layer is dried over magnesium sulfate and concentrated in vacuo. Crystallization from ethyl acetate gives the title compound. M.p. 196-198° C.

### 2. 2-(4-Benzyloxy-phenyl)-6-(3,4-dimethoxy-phenyl)-4,4-dimethyl-4,5-dihydro-2H-pyridazin-3-one

The title compound is prepared analogous as described for Example 1 using 100 mmol of intermediate A1 and 100 mmol of 4-benzyloxyphenylhydrazine. Crystallisation from diethyl ether gives the title compound. M.p. 137-138° C.

### 3. 6-(3,4-Dimethoxy-phenyl)-4,4-dimethyl-2-(4-{1-[4-(2-morpholin-4-yl-ethyl)-piperazin-1-yl]-methanoyl}-phenyl)-4,5-dihydro-2H-pyridazin-3-one dihydrochloride

7 mmol of (3-dimethylamino-propyl)-ethyl-carbodiimide is added to a solution of 5 mmol of compound 1 and 5 mmol of 4-(2-piperazin-1-yl-ethyl)-morpholine in 20 ml of dimethylformamide. The resulting solution is stirred for 2 h at RT and subsequently evaporated. The residue is dissolved in ethyl acetate and this solution is washed twice with aqueous sodium carbonate. The organic layer is dried over magnesium sulfate and evaporated. The residue is purified by chromatography (elution with ethyl acetate:methanol/1:1). The fractions containing the product are collected and evaporated. The residue is dissolved in ethanol. Addition of a saturated solution of hydrochloric acid in diethyl ether causes precipitation of the title compound. The precipitate is recrystallised from ethanol. M.p. 249° C., (decomposition).

### 4. 6-(3,4-Dimethoxy-phenyl)-4,4-dimethyl-2-{4-[1-(4-pyridin-4-yl-piperazin-1-yl)-methanoyl]-phenyl}-4,5-dihydro-2H-pyridazin-3-one dihydrochloride

The title compound is prepared analogous as described for Example 3 using compound 1 and 4-pyridylpiperazine. M.p. 223-225° C.

### 5. 6-(3,4-Dimethoxy-phenyl)-2-(4-hydroxy-phenyl)-4,4-dimethyl-4,5-dihydro-2H-pyridazin-3-one

A mixture of 30 mmol of compound 2.120 mmol of ammonium formate and 1 g of Pd/C (5%), in 150 ml of ethanol is refluxed for 30 min. After filtering the solution, the solvent is evaporated and the residue is dissolved in dichloromethane. After filtering the solution, the solvent is evaporated and the title compound is crystallised from diethyl ether. M.p. 176-178° C.

### 6. 6-(3,4-Dimethoxy-phenyl)-4,4-dimethyl-2-[4-(1-morpholin-4-yl-methanoyl)-phenyl]-4,5-dihydro-2H-pyridazin-3-one

The title compound is prepared analogous as described for Example 3 using compound 1 and morpholine. Crystallisation from ethyl acetate gives the title compound. M.p. 165-166° C.

### 7. 2-{4-[3-(3,4-Dimethoxy-phenyl)-5,5-dimethyl-6-oxo-5,6-dihydro-4H-pyridazin-1-yl]-phenoxy}-acetamide

A mixture of 5 mmol of compound 5, 7 mmol of 2-chloroacetamide and 10 mmol of potassium carbonate in 20 ml of dimethylformamide is stirred for 2 h at 70° C. After cooling to RT, the mixture is diluted with ethyl acetate and filtered. The solvent is evaporated and the residue purified by chromatography (elution with ethyl acetate). Crystallisation from diethyl ether gives the title compound. M.p. 184-187° C.

### 8. 6-(3,4-Dimethoxy-phenyl)-4,4-dimethyl-2-(4-{1-[4-(2-oxo-2-pyrrolidin-1-yl-ethyl)-piperazin-1-yl]-methanoyl}-phenyl)-4,5-dihydro-2H-pyridazin-3-one hydrochloride

The title compound is prepared analogous as described for Example 3 using compound 1 and 2-piperazin-1-yl-1-pyrrolidin-1-yl-ethanone. M.p. 144-146° C.

### 9. 6-(3,4-Dimethoxy-phenyl)-4,4-dimethyl-2-{4-[2-(4-methyl-piperazin-1-yl)-2-oxo-ethoxy]-phenyl}-4,5-dihydro-2H-pyridazin-3-one hydrochloride

Prepared from compound 5 and 2-chloro-1-(4-methyl-piperazin-1-yl)-ethanone as described for compound 7. The crude product is purified by chromatography (elution with ethyl acetate:methanol:triethylamine/4:1:1). The title compound is crystallized as the hydrochloride from ethyl acetate by the addition of a saturated solution of hydrochloric acid in diethyl ether. M.p. 198-202° C.

### 10. 6-(3,4-Dimethoxy-phenyl)-4,4-dimethyl-2-[4-(2-morpholin-4-yl-2-oxo-ethoxy)-phenyl]-4,5-dihydro-2Hpyridazin-3-one

The title compound is prepared analogous as described for Example 7 using compound 5 and 2-chloro-1-morpholin-4-yl-ethanone. M.p. 74-79° C.

### 11. 3-[3-(3,4-Dimethoxy-phenyl)-5,5-dimethyl-6-oxo-5,6-dihydro-4H-pyridazin-1-yl]-benzoic acid

The title compound is prepared analogous as described for Example 1 using intermediate A1 and 3-hydrazinobenzoic acid as described for product 1. M.p. 165-167° C.

### 12. {4-[3-(3,4-Dimethoxy-phenyl)-5,5-dimethyl-6-oxo-5,6-dihydro-4H-pyridazin-1-yl]-phenyl}-acetic acid

The title compound is prepared analogous as described for Example 1 using intermediate A1 and 4-hydrazinophenyl acetic acid. M.p. 186-188° C.

### 13. 6-(3,4-Dimethoxy-phenyl)-4,4-dimethyl-2-[3-(1-morpholin-4-yl-methanoyl)-phenyl]-4,5-dihydro-2H-pyridazin-3-one

The title compound is prepared analogous as described for Example 3 using compound 11 and morpholine. M.p. 108-109° C.

### 14. 6-(3,4-Dimethoxy-phenyl)-4,4-dimethyl-2-[4-(2-morpholin-4-yl-2-oxo-ethyl)-phenyl]-4,5-dihydro-2H-pyridazin-3-one

The title compound is prepared analogous as described for Example 3 using compound 12 and morpholine. M.p. 165-167° C.

### 15. 2-(4-Bromo-phenyl)-6-(3,4-dimethoxy-phenyl)-4,4-dimethyl-4,5-dihydro-2H-pyridazin-3-one

The title compound is prepared analogous as described for Example 1 using intermediate A1 and (4-Bromo-phenyl)-hydrazine. M.p. 123-124° C.

### 16. 6-(3,4-Dimethoxy-phenyl)-4,4-dimethyl-2-(4-morpholin-4-yl-phenyl)-4,5-dihydro-2H-pyridazin-3-one

A mixture of 1.2 mmol of compound 15, 1.3 mmol of morpholine, 6.2 mg of Pd[P(t-Bu)3]2, 2.2 mg of cetyltrimethylammonium bromide, 1.5 mmol of potassium hydroxide and 1.5 mmol of water in 1 ml of dioxane is heated, under thorough exclusion of oxygen, for 40 h at 85° C. After cooling to room temperature the mixture is diluted with ethyl acetate and washed with water. The ethyl acetate solution is dried over magnesium sulfate and evaporated. The residue is purified by chromatography [elution with a mixture (1/1; vol/vol) of ethyl acetate and petroleum ether (60-80° C.). The title compound is dissolved in a minimal amount of dichloromethane and diethyl ether is added until crystallization started. M.p. 178-179.

### Starting Compounds and Intermediates

### A1. 4-(3,4-dimethoxyphenyl)-2,2-dimethyl-4-oxo-butyric acid

Under an atmosphere of dry nitrogen a grignard solution, prepared from 43.4 g 3,4-dimethoxybromo-benzene and 6.1 g magnesium in 200 ml of tetrahydrofurane, is added dropwise to a solution of 20.5 g 3,3-dimethyl-dihydro-furan-2,5-dione in 200 ml of tetrahydrofurane cooled in an icebath. The reaction mixture is stirred for an additional hour at RT. 100 ml of a 20% ammonium chloride solution is added and the water layer is extracted twice with 75 ml of ethyl acetate. The combined organic layers are washed twice with 100 ml of half saturated brine and extracted with 3×100 mL 1M sodium hydroxide solution. The aqueous layers are washed with 75 ml of ethyl acetate, acidified with concentrated hydrochloric acid and extracted 3 times with 100 ml of dichloromethane. The organic layers are dried over magnesium sulfate, filtered and concentrated in vacuo. The oily residue is crystallized from ethyl acetate/petroleum ether (60-80° C.). M.p. 114-116° C.

### COMMERCIAL UTILITY

The compounds according to the invention have useful pharmacological properties which make them industrially utilizable. As selective cyclic nucleotide phosphodiesterase (PDE) inhibitors (specifically of type 4), they are suitable on the one hand as bronchial therapeutics (for the treatment of airway obstructions on account of their dilating action but also on account of their respiratory rate or respiratory drive-increasing action) and for the removal of erectile dysfunction on account of their vascular dilating action, but on the other hand especially for the treatment of disorders, in particular of an inflammatory nature, e.g. of the airways (asthma prophylaxis), of the skin, of the intestine, of the eyes, of the CNS and of the joints, which are mediated by mediators such as histamine, PAF (platelet-activating factor), arachidonic acid derivatives such as leukotrienes and prostaglandins, cytokines, interleukins, chemokines, alpha-, beta- and gamma-interferon, tumor necrosis factor (TNF) or oxygen free radicals and proteases. In this context, the compounds according to the invention are distinguished by a low toxicity, a good enteral absorption (high bioavailability), a large therapeutic breadth and the absence of significant side effects.

On account of their PDE-inhibiting properties, the compounds according to the invention can be employed in human and veterinary medicine as therapeutics, where they can be used, for example, for the treatment and prophylaxis of the following illnesses: acute and chronic (in particular inflammatory and allergen-induced) airway disorders of varying origin (bronchitis, allergic bronchitis, bronchial asthma, emphysema, COPD); dermatoses (especially of proliferative, inflammatory and allergic type) such as psoriasis (vulgaris), toxic and allergic contact eczema, atopic eczema, seborrhoeic eczema, Lichen simplex, sunburn, pruritus in the anogenital area, alopecia greata, hypertrophic scars, discoid lupus erythematosus, follicular and widespread pyodermias, endogenous and exogenous acne, acne rosacea and other proliferative, inflammatory and allergic skin disorders; disorders which are based on an excessive release of TNF and leukotrienes, for example disorders of the arthritis type (rheumatoid arthritis, rheumatoid spondylitis, osteoarthritis and other arthritic conditions), disorders of the immune system (AIDS, multiple sclerosis), graft versus host reaction, allograft rejections, types of shock (septic shock, endotoxin shock, gram-negative sepsis, toxic shock syndrome and ARDS (adult respiratory distress syndrome)) and also generalized inflammations in the gastrointestinal region (Crohn's disease and ulcerative colitis); disorders which are based on allergic and/or chronic, immunological false reactions in the region of the upper airways (pharynx, nose) and the adjacent regions (paranasal sinuses, eyes), such as allergic rhinitis/sinusitis, chronic rhinitis/sinusitis, allergic conjunctivitis and also nasal polyps; but also disorders of the heart which can be treated by PDE inhibitors, such as cardiac insufficiency, or disorders which can be treated on account of the tissue-relaxant action of the PDE inhibitors, such as, for example, erectile dysfunction or colics of the kidneys and of the ureters in connection with kidney stones. In addition, the compounds of the invention are useful in the treatment of diabetes insipidus, diabetes mellitus, leukaemia, osteoporosis and conditions associated with cerebral metabolic inhibition, such as cerebral senility, senile dementia (Alzheimer's disease), memory impairment associated with Parkinson's disease or multi-infarct dementia; and also illnesses of the central nervous system, such as depressions or arteriosclerotic dementia.

The invention further relates to a method for the treatment of mammals, including humans, which are suffering from one of the above mentioned illnesses. The method is characterized in that a therapeutically active and pharmacologically effective and tolerable amount of one or more of the compounds according to the invention is administered to the ill mammal.

The invention further relates to the compounds according to the invention for use in the treatment and/or prophylaxis of illnesses, especially the illnesses mentioned.

The invention also relates to the use of the compounds according to the invention for the production of pharmaceutical compositions which are employed for the treatment and/or prophylaxis of the illnesses mentioned.

The invention furthermore relates to pharmaceutical compositions for the treatment and/or prophylaxis of the illnesses mentioned, which contain one or more of the compounds according to the invention.

Additionally, the invention relates to an article of manufacture, which comprises packaging material and a pharmaceutical agent contained within said packaging material, wherein the pharmaceutical agent is therapeutically effective for antagonizing the effects of the cyclic nucleotide phosphodiesterase of type 4 (PDE4), ameliorating the symptoms of an PDE4-mediated disorder, and wherein the packaging material comprises a label or package insert which indicates that the pharmaceutical agent is useful for preventing or treating PDE4-mediated disorders, and wherein said pharmaceutical agent comprises one or more compounds of formula 1 according to the invention. The packaging material, label and package insert otherwise parallel or resemble what is generally regarded as standard packaging material, labels and package inserts for pharmaceuticals having related utilities.

The pharmaceutical compositions are prepared by processes which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries or excipients which are suitable for the desired pharmaceutical formulations on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

The administration of the pharmaceutical compositions according to the invention may be performed in any of the generally accepted modes of administration available in the art. Illustrative examples of suitable modes of administration include intravenous, oral, nasal, parenteral, topical, transdermal and rectal delivery. Oral delivery is preferred.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatoses, the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the active compounds is carried out in the order of magnitude customary for PDE inhibitors. Topical application forms (such as ointments) for the treatment of dermatoses thus contain the active compounds in a concentration of, for example, 0.1-99%. The dose for administration by inhalation is customarily between 0.1 and 3 mg per day. The customary dose in the case of systemic therapy (p.o. or i.v.) is between 0.03 and 3 mg/kg per day.

### Biological Investigations

The second messenger cyclic AMP (cAMP) is well-known for inhibiting inflammatory and immunocompetent cells. The PDE4 isoenzyme is broadly expressed in cells involved in the initiation and propagation of inflammatory diseases (H Tenor and C Schudt, in “Phosphodiesterase Inhibitors”, 21-40, “The Handbook of Immunopharmacology”, Academic Press, 1996), and its inhibition leads to an increase of the intracellular cAMP concentration and thus to the inhibition of cellular activation (J E Souness et al., Immunopharmacology 47:127-162, 2000).

The antiinflammatory potential of PDE4 inhibitors in vivo in various animal models has been described (M M Teixeira, TiPS 18:164-170, 1997). For the investigation of PDE4 inhibition on the cellular level (in vitro), a large variety of proinflammatory responses can be measured. Examples are the superoxide production of neutrophilic (C Schudt et al., Arch Pharmacol 344: 682-690, 1991) or eosinophilic (A Hatzelmann et al., Brit J Pharmacol 114: 821-831, 1995) granulocytes, which can be measured as luminol-enhanced chemiluminescence, or the synthesis of tumor necrosis factor-α in monocytes, macrophages or dendritic cells (Gantner et al., Brit J Pharmacol 121: 221-231, 1997, and Pulmonary Pharmacol Therap 12: 377-386, 1999). In addition, the immunomodulatory potential of PDE4 inhibitors is evident from the inhibition of T-cell responses like cytokine synthesis or proliferation (D M Essayan, Biochem Pharmacol 57: 965-973, 1999). Substances which inhibit the secretion of the afore-mentioned proinflammatory mediators are those which inhibit PDE4. PDE4 inhibition by the compounds according to the invention is thus a central indicator for the suppression of inflammatory processes.

### Method for Measuring Inhibition of PDE4 Activities

PDE4B2 (GB no. M97515) was a gift of Prof. M. Conti (Stanford University, USA). It was amplified from the original plasmid (pCMV5) via PCR with primers Rb9 (5′-GCCAGCGTGCAAATAATGAAGG-3′) and Rb10 (5′-AGAGGGGGATTATGTATCCAC-3′) and cloned into the pCR-Bac vector (Invitrogen, Groningen, NL).

The recombinant baculovirus was prepared by means of homologous recombination in SF9 insect cells. The expression plasmids were cotransfected with Bac-N-Blue (Invitrogen, Groningen, NL) or Baculo-Gold DNA (Pharmingen, Hamburg) using a standard protocol (Pharmingen, Hamburg). Wt virus-free recombinant virus supernatants were selected using plaque assay methods. After that, high-titre virus supernatants were prepared by amplifying 3 times. PDE4B2 was expressed in SF21 cells by infecting 2×10⁶ cells/ml with an MOI (multiplicity of infection) between 1 and 10 in serum-free SF900 medium (Life Technologies, Paisley, UK). The cells were cultured at 28° C. for 48-72 hours, after which they were pelleted for 5-10 min at 1000 g and 4° C.

The SF21 insect cells were resuspended, at a concentration of approx. 10⁷ cells/ml, in ice-cold (4° C.) homogenization buffer (20 mM Tris, pH 8.2, containing the following additions: 140 mM NaCl, 3.8 mM KCl, 1 mM EGTA, 1 mM MgCl₂, 10 mM β-mercaptoethanol, 2 mM benzamidine, 0.4 mM Pefablock, 10 μM leupeptin, 10 μM pepstatin A, 5 μM trypsin inhibitor) and disrupted by ultrasonication. The homogenate was then centrifuged for 10 min at 1000×g and the supernatant was stored at −80° C. until sub-sequent use (see below). The protein content was determined by the Bradford method (BioRad, Munich) using BSA as the standard.

PDE4B2 activity was inhibited by the compounds according to the invention in a modified SPA (scintillation proximity assay) test, supplied by Amersham Biosciences (see procedural instructions “phosphodiesterase [3H]cAMP SPA enzyme assay, code TRKQ 7090”), carried out in 96-well microtitre plates (MTP's). The test volume is 100 μl and contains 20 mM Tris buffer (pH 7.4), 0.1 mg of BSA (bovine serum albumin)/ml, 5 mM Mg²⁺, 0.5 μM cAMP (including about 50,000 cpm of [3H]cAMP), 1 μl of the respective substance dilution in DMSO and sufficient recombinant PDE (1000×g supernatant, see above) to ensure that 10-20% of the cAMP is converted under the said experimental conditions. The final concentration of DMSO in the assays (1% v/v) does not substantially affect the activity of the PDEs investigated. After a preincubation of 5 min at 37° C., the reaction is started by adding the substrate (cAMP) and the assays are incubated for a further 15 min; after that, they are stopped by adding SPA beads (50 μl). In accordance with the manufacturer's instructions, the SPA beads had previously been resuspended in water, but were then diluted 1:3 (v/v) in water; the diluted solution also contains 3 mM IBMX to ensure a complete PDE activity stop. After the beads have been sedimented (>30 min), the MTP's are analyzed in commercially available luminescence detection devices. The corresponding IC₅₀ values of the compounds for the inhibition of PDE4B2 activity are determined from the concentration-effect curves by means of non-linear regression.

The inhibitory values determined for the compounds according to the invention follow from the following Table 1, in which the numbers of the compounds correspond to the numbers of the examples.

TABLE 1
Inhibition of PDE4 acitivity [measured as −logIC₅₀ (mol/l)]

| Compound | PDE4 Inhibition |
|---|---|
| 1 | 7.49 |
| 2 | 8.40 |
| 3 | 7.95 |
| 4 | 8.11 |
| 5 | 8.22 |
| 6 | 8.76 |
| 7 | 7.98 |
| 8 | 8.12 |
| 9 | 8.06 |
| 10 | 7.81 |

1. A compound of formula 1 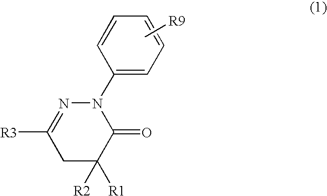
in which
R1 is 1-4C-alkyl and
R2 is 1-4C-alkyl,
R3 represents a phenyl derivative of formulae (a) or (b) 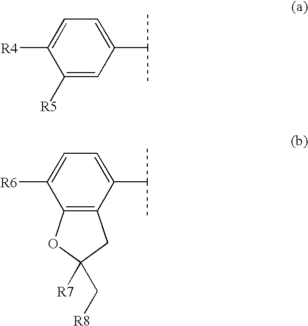
wherein
R4 is 1-4C-alkoxy or 1-4C-alkoxy which is completely or predominantly substituted by fluorine,
R5 is 1-8C-alkoxy, 3-7C-cycloalkoxy, 3-7C-cycloalkylmethoxy, or 1-4C-alkoxy which is completely or predominantly substituted by fluorine,
R6 is 1-4C-alkoxy, 3-5C-cycloalkoxy, 3-5C-cycloalkylmethoxy, or 1-4C-alkoxy which is completely or predominantly substituted by fluorine,
R7 is 1-4C-alkyl and
R8 is hydrogen or 1-4C-alkyl,
or wherein
R7 and R8 together and with inclusion of the two carbon atoms, to which they are bonded, form a spiro-linked 5-, 6- or 7-membered hydrocarbon ring, optionally interrupted by an oxygen or sulphur atom,
R9 is hydroxyl, halogen, nitro, cyano, 1-4C-alkyl, trifluoromethyl, 1-4C-alkoxy, 1-4C-alkoxy which is completely or predominantly substituted by fluorine, hydroxycarbonyl, hydroxycarbonyl-1-4C-alkyl, 1-4C-alkoxycarbonyl, 1-4C-alkylcarbonyl, 1-4C-alkylcarbonylamino, 1-4C-alkylcarbonyloxy, 1-4C-alkylsulfonyl, benzyloxy, —C(O)R10, —S(O)₂—R11, —O—(CH₂)ₙ—C(O)—R12, —(CH₂)ᵣ—C(O)—R26 or —N(R29)R30,
R10 is —N(R13)R14,
R11 is —N(R22)R23,
R12 is —N(R24)R25,
R13 is hydrogen, 1-4C-alkyl, 3-7C-cycloalkyl or 3-7C-cycloalkylmethyl,
R14 is hydrogen, 1-4C-alkyl, 3-7C-cycloalkyl or 3-7C-cycloalkylmethyl,
or R13 and R14 together and with inclusion of the nitrogen atom to which they are bonded, form a 1-pyrrolidinyl-, 1-piperidinyl-, 1-hexahydroazepinyl-ring or a ring of formula (c), 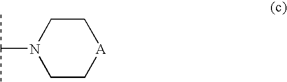
wherein
A is O, S, SO, SO₂ or NR15,
R15 is hydrogen, 1-4C-alkyl, phenyl, pyridyl, —(CH₂)ₘ—R16 or —(CH₂)ₚ—C(O)R17,
R16 is —N(R18)R19,
R17 is —N(R20)R21,
R18 is hydrogen, 1-4C-alkyl, 3-7C-cycloalkyl or 3-7C-cycloalkylmethyl,
R19 is hydrogen, 1-4C-alkyl, 3-7C-cycloalkyl or 3-7C-cycloalkylmethyl,
or R18 and R19 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl-, 4-thiomorpholinyl-, thiomorpholin-1-oxide-4-yl- or thiomorpholin-1,1-dioxide-4-yl-ring,
R20 is hydrogen, 1-4C-alkyl, 3-7C-cycloalkyl or 3-7C-cycloalkylmethyl,
R21 is hydrogen, 1-4C-alkyl, 3-7C-cycloalkyl or 3-7C-cycloalkylmethyl,
or R20 and R21 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R22 is hydrogen, 1-4C-alkyl, 3-7C-cycloalkyl or 3-7C-cycloalkylmethyl,
R23 is hydrogen, 1-4C-alkyl, 3-7C-cycloalkyl or 3-7C-cycloalkylmethyl,
or R22 and R23 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R24 is hydrogen, 1-4C-alkyl, 3-7C-cycloalkyl or 3-7C-cycloalkylmethyl,
R25 is hydrogen, 1-4C-alkyl, 3-7C-cycloalkyl or 3-7C-cycloalkylmethyl,
or R24 and R25 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R26 is —N(R27)R28,
R27 is hydrogen, 1-4C-alkyl, 3-7C-cycloalkyl or 3-7C-cycloalkylmethyl,
R28 is hydrogen, 1-4C-alkyl, 3-7C-cycloalkyl or 3-7C-cycloalkylmethyl,
or R27 and R28 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R29 is hydrogen, 1-4C-alkyl, 3-7C-cycloalkyl or 3-7C-cycloalkylmethyl,
R30 is hydrogen, 1-4C-alkyl, 3-7C-cycloalkyl or 3-7C-cycloalkylmethyl,
or R29 and R30 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
n is an integer from 1 to 2,
m is an integer from 2 to 4,
p is an integer from 1 to 4,
r is an integer from 1 to 4,
or a salt thereof.

2. A compound of formula 1 according to claim 1, in which
R1 is 1-4C-alkyl,
R2 is 1-4C-alkyl,
R3 represents a phenyl derivative of formulae (a) or (b) 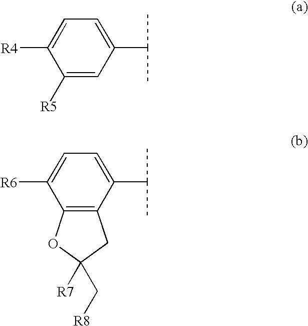
wherein
R4 is 1-2C-alkoxy or 1-2C-alkoxy which is completely or predominantly substituted by fluorine,
R5 is 1-2C-alkoxy or 1-2C-alkoxy which is completely or predominantly substituted by fluorine,
R6 is 1-2C-alkoxy or 1-2C-alkoxy which is completely or predominantly substituted by fluorine,
R7 is methyl and
R8 is hydrogen,
or wherein
R7 and R8 together and with inclusion of the two carbon atoms, to which they are bonded, form a spiro-linked cyclopentane, cyclohexane, tetrahydrofuran or tetrahydropyran ring,
R9 is hydroxyl, halogen, hydroxycarbonyl, hydroxycarbonyl-1-4C-alkyl, benzyloxy, —C(O)R10, —S(O)₂—R11, —O—(CH₂)ₙ—C(O)—R12, —(CH₂)ᵣ—C(O)—R26 or —N(R29)R30,
R10 is —N(R13)R14,
R11 is —N(R22)R23,
R12 is —N(R24)R25,
R13 is hydrogen or 1-4C-alkyl,
R14 is hydrogen or 1-4C-alkyl,
or R13 and R14 together and with inclusion of the nitrogen atom to which they are bonded, form a 1-pyrrolidinyl-, 1-piperidinyl-, 1-hexahydroazepinyl-ring or a ring of formula (c), 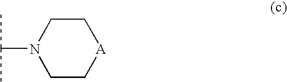
wherein
A is O, S, SO, SO₂ or NR15,
R15 is hydrogen, 1-4C-alkyl, phenyl, pyridyl, —(CH₂)ₘ—R16 or —(CH₂)ₚ—C(O)R17,
R16 is —N(R18)R19,
R17 is —N(R20)R21,
R18 is hydrogen or 1-4C-alkyl,
R19 is hydrogen or 1-4C-alkyl,
or R18 and R19 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl-, 4-thiomorpholinyl-, thiomorpholin-1-oxide-4-yl- or thiomorpholin-1,1-dioxide-4-yl-ring,
R20 is hydrogen or 1-4C-alkyl,
R21 is hydrogen or 1-4C-alkyl,
or R20 and R21 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R22 is hydrogen or 1-4C-alkyl,
R23 is hydrogen or 1-4C-alkyl,
or R22 and R23 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R24 is hydrogen or 1-4C-alkyl,
R25 is hydrogen or 1-4C-alkyl,
or R24 and R25 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R26 is —N(R27)R28,
R27 is hydrogen or 1-4C-alkyl,
R28 is hydrogen or 1-4C-alkyl,
or R27 and R28 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R29 is hydrogen or 1-4C-alkyl,
R30 is hydrogen or 1-4C-alkyl,
or R29 and R30 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
n is an integer from 1 to 2,
m is an integer from 2 to 4,
p is an integer from 1 to 4,
r is an integer from 1 to 4,
or a salt thereof.

3. A compound of formula 1 according to claim 1, in which
R1 is methyl or ethyl,
R2 is methyl or ethyl,
R3 represents a phenyl derivative of formulae (a) or (b) 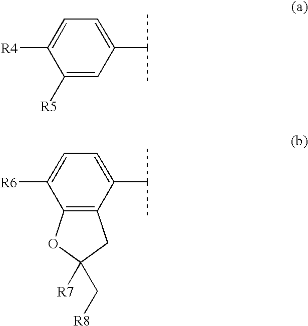
wherein
R4 is methoxy, ethoxy or difluoromethoxy,
R5 is methoxy, ethoxy or difluoromethoxy,
R6 is methoxy, ethoxy or difluoromethoxy,
R7 is methyl and
R8 is hydrogen,
or wherein
R7 and R8 together and with inclusion of the two carbon atoms, to which they are bonded, form a spiro-linked cyclopentane, cyclohexane, tetrahydrofuran or tetrahydropyran ring,
R9 is hydroxyl, halogen, hydroxycarbonyl, hydroxycarbonylmethyl or benzyloxy,
or a salt thereof.

4. A compound of formula 1 according to claim 1, in which
R1 is methyl or ethyl,
R2 is methyl or ethyl,
R3 represents a phenyl derivative of formulae (a) or (b) 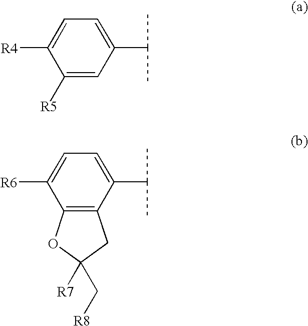
wherein
R4 is methoxy, ethoxy or difluoromethoxy,
R5 is methoxy, ethoxy or difluoromethoxy,
R6 is methoxy, ethoxy or difluoromethoxy,
R7 is methyl and
R8 is hydrogen,
or wherein
R7 and R8 together and with inclusion of the two carbon atoms, to which they are bonded, form a spiro-linked cyclopentane, cyclohexane, tetrahydrofuran or tetrahydropyran ring,
R9 is —C(O)R10,
R10 is —N(R13)R14,
R13 is hydrogen or 1-4C-alkyl,
R14 is hydrogen or 1-4C-alkyl,
or R13 and R14 together and with inclusion of the nitrogen atom to which they are bonded, form a 1-pyrrolidinyl-, a 1-piperidinyl-ring or a ring of formula (c), 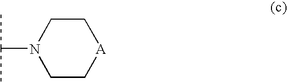
wherein
A is O, S, SO₂ or NR15,
R15 is 1-4C-alkyl, phenyl, pyridyl, —(CH₂)_{R}16 or —(CH₂)ₚ—C(O)R17,
R16 is —N(R18)R19,
R17 is —N(R20)R21,
R18 is hydrogen or 1-4C-alkyl,
R19 is hydrogen or 1-4C-alkyl,
or R18 and R19 together and with inclusion of the nitrogen atom to which they are bonded, form a 1-pyrrolidinyl-, 1-piperidinyl-, 1-methyl-piperazin-4-yl- or a 4-morpholinyl-ring,
R20 is hydrogen or 1-4C-alkyl,
R21 is hydrogen or 1-4C-alkyl,
or R20 and R21 together and with inclusion of the nitrogen atom to which they are bonded, form a 1-pyrrolidinyl-, 1-piperidinyl-, 1-methyl-piperazin-4-yl- or a 4-morpholinyl-ring,
m is 2,
p is 1,
or a salt thereof.

5. A compound of formula 1 according to claim 1, in which
R1 is methyl or ethyl,
R2 is methyl or ethyl,
R3 represents a phenyl derivative of formulae (a) or (b) 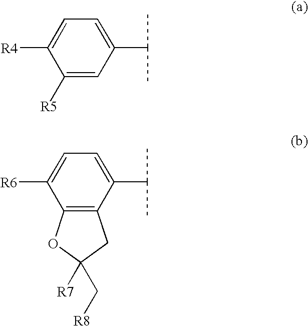
wherein
R4 is methoxy, ethoxy or difluoromethoxy,
R5 is methoxy, ethoxy or difluoromethoxy,
R6 is methoxy, ethoxy or difluoromethoxy,
R7 is methyl and
R8 is hydrogen,
or wherein
R7 and R8 together and with inclusion of the two carbon atoms, to which they are bonded, form a spiro-linked cyclopentane, cyclohexane, tetrahydrofuran or tetrahydropyran ring,
R9 is —S(O)₂—R11,
R11 is —N(R22)R23,
R22 is hydrogen or 1-4C-alkyl,
R23 is hydrogen or 1-4C-alkyl,
or R22 and R23 together and with inclusion of the nitrogen atom to which they are bonded, form a 1-pyrrolidinyl-, 1-piperidinyl-, 1-methyl-piperazin-4-yl- or a 4-morpholinyl-ring,
or a salt thereof.

6. A compound of formula 1 according to claim 1, in which
R1 is methyl or ethyl,
R2 is methyl or ethyl,
R3 represents a phenyl derivative of formulae (a) or (b) 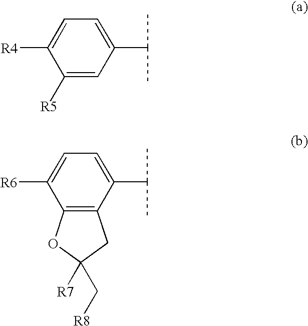
wherein
R4 is methoxy, ethoxy or difluoromethoxy,
R5 is methoxy, ethoxy or difluoromethoxy,
R6 is methoxy, ethoxy or difluoromethoxy,
R7 is methyl and
R8 is hydrogen,
or wherein
R7 and R8 together and with inclusion of the two carbon atoms, to which they are bonded, form a spiro-linked cyclopentane, cyclohexane, tetrahydrofuran or tetrahydropyran ring,
R9 is —O— (CH₂)ₙ—C(O)—R12,
R12 is —N(R24)R25,
R24 is hydrogen or 1-4C-alkyl,
R25 is hydrogen or 1-4C-alkyl,
or R24 and R25 together and with inclusion of the nitrogen atom to which they are bonded, form a 1-pyrrolidinyl-, 1-piperidinyl-, 1-methyl-piperazin-4-yl- or a 4-morpholinyl-ring,
n is 1,
or a salt thereof.

7. A compound of formula 1 according to claim 1, in which
R1 is methyl or ethyl,
R2 is methyl or ethyl,
R3 represents a phenyl derivative of formulae (a) or (b) 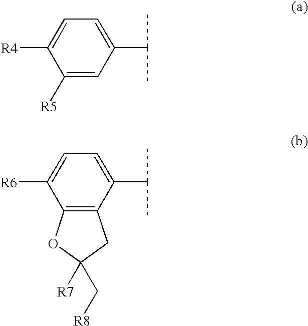
wherein
R4 is methoxy, ethoxy or difluoromethoxy,
R5 is methoxy, ethoxy or difluoromethoxy,
R6 is methoxy, ethoxy or difluoromethoxy,
R7 is methyl and
R8 is hydrogen,
or wherein
R7 and R8 together and with inclusion of the two carbon atoms, to which they are bonded, form a spiro-linked cyclopentane, cyclohexane, tetrahydrofuran or tetrahydropyran ring,
R9 is —(CH₂)ᵣ—C(O)—R26,
R26 is —N(R27)R28,
R27 is hydrogen or 1-4C-alkyl,
R28 is hydrogen or 1-4C-alkyl,
or R27 and R28 together and with inclusion of the nitrogen atom to which they are bonded, form a 1-pyrrolidinyl-, 1-piperidinyl-, 1-methyl-piperazin-4-yl- or a 4-morpholinyl-ring,
r is 1,
or a salt thereof.

8. A compound of formula 1 according to claim 1, in which
R1 is methyl or ethyl,
R2 is methyl or ethyl,
R3 represents a phenyl derivative of formulae (a) or (b) 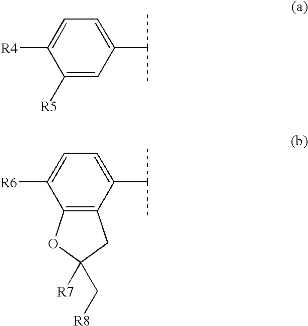
wherein
R4 is methoxy, ethoxy or difluoromethoxy,
R5 is methoxy, ethoxy or difluoromethoxy,
R6 is methoxy, ethoxy or difluoromethoxy,
R7 is methyl and
R8 is hydrogen,
or wherein
R7 and R8 together and with inclusion of the two carbon atoms, to which they are bonded, form a spiro-linked cyclopentane, cyclohexane, tetrahydrofuran or tetrahydropyran ring,
R9 is —N(R29)R30,
R29 and R30 together and with inclusion of the nitrogen atom to which they are bonded, form a 1-pyrrolidinyl-, 1-piperidinyl-, 1-methyl-piperazin-4-yl- or a 4-morpholinyl-ring,
or a salt thereof.

9. A compound of formula 1 according to claim 1, in which
R1 is 1-4C-alkyl and
R2 is 1-4C-alkyl,
R3 represents a phenyl derivative of formulae (a) or (b) 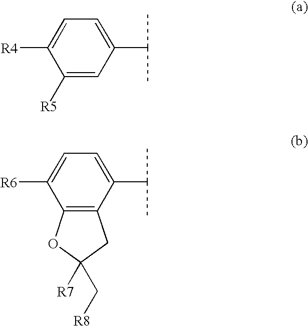
wherein
R4 is 1-4C-alkoxy or 1-4C-alkoxy which is completely or predominantly substituted by fluorine,
R5 is 1-8C-alkoxy, 3-7C-cycloalkoxy, 3-7C-cycloalkylmethoxy, or 1-4C-alkoxy which is completely or predominantly substituted by fluorine,
R6 is 1-4C-alkoxy, 3-5C-cycloalkoxy, 3-5C-cycloalkylmethoxy, or 1-4C-alkoxy which is completely or predominantly substituted by fluorine,
R7 is 1-4C-alkyl and
R8 is hydrogen or 1-4C-alkyl,
or wherein
R7 and R8 together and with inclusion of the two carbon atoms, to which they are bonded, form a spiro-linked 5-, 6- or 7-membered hydrocarbon ring, optionally interrupted by an oxygen or sulphur atom,
R9 is hydroxyl, halogen, nitro, cyano, hydroxycarbonyl, 1-4C-alkyl, trifluoromethyl, 1-4C-alkoxy, 1-4C-alkoxy which is completely or predominantly substituted by fluorine, amino, mono- or di-1-4C-alkylamino, 1-4C-alkylcarbonylamino, 1-4C-alkylcarbonyloxy, benzyloxy, —C(O)R10, —S(O)₂—R11 or —O—(CH₂)ₙ—C(O)—R12,
R10 is 1-4C-alkyl, 1-4C-alkoxy or —N(R13)R14,
R11 is 1-4C-alkyl or —N(R22)R23,
R12 is —N(R24)R25,
R13 is hydrogen, 1-4C-alkyl, 3-7C-cycloalkyl or 3-7C-cycloalkylmethyl,
R14 is hydrogen, 1-4C-alkyl, 3-7C-cycloalkyl or 3-7C-cycloalkylmethyl,
or R13 and R14 together and with inclusion of the nitrogen atom to which they are bonded, form a 1-pyrrolidinyl-, 1-piperidinyl-, 1-hexahydroazepinyl-ring or a ring of formula (c), 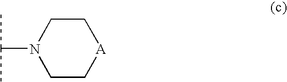
wherein
A is O, S, SO, SO₂ or NR15,
R15 is hydrogen, 1-4C-alkyl, phenyl, pyridyl, —(CH₂)ₘ—R16 or —(CH₂)ₚ—C(O)R17,
R16 is —N(R18)R19,
R17 is —N(R20)R21,
R18 is hydrogen, 1-4C-alkyl, 3-7C-cycloalkyl or 3-7C-cycloalkylmethyl,
R19 is hydrogen, 1-4C-alkyl, 3-7C-cycloalkyl or 3-7C-cycloalkylmethyl,
or R18 and R19 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl-, 4-thiomorpholinyl-, thiomorpholin-1-oxide-4-yl- or thiomorpholin-1,1-dioxide-4-yl-ring,
R20 is hydrogen, 1-4C-alkyl, 3-7C-cycloalkyl or 3-7C-cycloalkylmethyl,
R21 is hydrogen, 1-4C-alkyl, 3-7C-cycloalkyl or 3-7C-cycloalkylmethyl,
or R20 and R21 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R22 is hydrogen, 1-4C-alkyl, 3-7C-cycloalkyl or 3-7C-cycloalkylmethyl,
R23 is hydrogen, 1-4C-alkyl, 3-7C-cycloalkyl or 3-7C-cycloalkylmethyl,
or R22 and R23 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R24 is hydrogen, 1-4C-alkyl, 3-7C-cycloalkyl or 3-7C-cycloalkylmethyl,
R25 is hydrogen, 1-4C-alkyl, 3-7C-cycloalkyl or 3-7C-cycloalkylmethyl,
or R24 and R25 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
n is an integer from 1 to 2,
m is an integer from 2 to 4,
p is an integer from 1 to 4,
or a salt thereof.

10. A compound of formula 1 according to claim 1, in which
R1 is 1-4C-alkyl,
R2 is 1-4C-alkyl,
R3 represents a phenyl derivative of formulae (a) or (b) 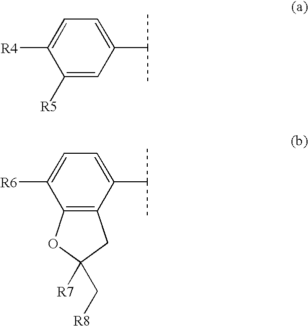
wherein
R4 is 1-2C-alkoxy or 1-2C-alkoxy which is completely or predominantly substituted by fluorine,
R5 is 1-4C-alkoxy,
R6 is 1-2C-alkoxy or 1-2C-alkoxy which is completely or predominantly substituted by fluorine,
R7 is methyl and
R8 is hydrogen,
or wherein
R7 and R8 together and with inclusion of the two carbon atoms, to which they are bonded, form a spiro-linked cyclopentane, cyclohexane, tetrahydrofurane or tetrahydropyran ring,
R9 is hydroxyl, hydroxycarbonyl, benzyloxy, —C(O)R10 or —O—(CH₂)ₙ—C(O)—R12,
R10 is —N(R13)R14,
R12 is —N(R24)R25,
R13 is hydrogen or 1-4C-alkyl,
R14 is hydrogen or 1-4C-alkyl,
or R13 and R14 together and with inclusion of the nitrogen atom to which they are bonded, form a 1-pyrrolidinyl-, 1-piperidinyl-, 1-hexahydroazepinyl-ring or a ring of formula (c), 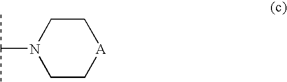
wherein
A is O, S, SO, SO₂ or NR15,
R15 is hydrogen, 1-4C-alkyl, phenyl, pyridyl, —(CH₂)ₘ—R16 or —(CH₂)ₚ—C(O)R17,
R16 is —N(R18)R19,
R17 is —N(R20)R21,
R18 is hydrogen or 1-4C-alkyl,
R19 is hydrogen or 1-4C-alkyl,
or R18 and R19 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl-, 4-thiomorpholinyl-, thiomorpholin-1-oxide-4-yl- or thiomorpholin-1,1-dioxide-4-yl-ring,
R20 is hydrogen or 1-4C-alkyl,
R21 is hydrogen or 1-4C-alkyl,
or R20 and R21 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
R24 is hydrogen or 1-4C-alkyl,
R25 is hydrogen or 1-4C-alkyl,
or R24 and R25 together and with inclusion of the nitrogen atom to which they are bonded, form a 1-pyrrolidinyl-, 1-piperidinyl-, 1-piperazinyl, 1-(1-4C-alkyl)-piperazin-4-yl-, 1-hexahydroazepinyl-, 4-morpholinyl, 4-thiomorpholinyl-, thiomorpholin-1-oxide-4-yl- or thiomorpholin-1,1-dioxide-4-yl-ring,
n is an integer from 1 to 2,
m is an integer from 2 to 4,
p is an integer from 1 to 4,
or a salt thereof.

11. A compound of formula 1 according to claim 1, in which
R1 is methyl,
R2 is methyl,
R3 represents a phenyl derivative of formula (a) 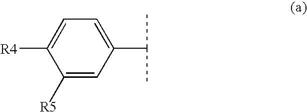
wherein
R4 is methoxy or ethoxy,
R5 is methoxy or ethoxy,
R9 is hydroxyl, hydroxycarbonyl, benzyloxy, —C(O)R10 or —O—(CH₂)ₙ—C(O)—R12,
R10 is —N(R13)R14,
R12 is —N(R24)R25,
R13 and R14 together and with inclusion of the nitrogen atom to which they are bonded, form a ring of formula (c), 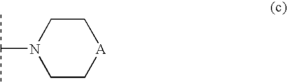
wherein
A is O or NR15,
R15 is pyrid-4-yl, —(CH₂)ₘ—R16 or —(CH₂)ₚ—C(O)R17,
R16 is 4-morpholinyl,
R17 is 1-pyrrolidinyl,
R24 is hydrogen,
R25 is hydrogen,
or R24 and R25 together and with inclusion of the nitrogen atom to which they are bonded, form a 1-methyl-piperazin-4-yl- or 4-morpholinyl-ring,
n is 1,
m is 2,
p is 1,
or a salt thereof.

12. (canceled)

13. A pharmaceutical composition comprising one or more compounds according to claim 1, or a pharmaceutically acceptable salt thereof, together with a pharmaceutically acceptable auxiliary and/or carrier.

14. (canceled)

15. A method for treating an illness treatable by the administration of a PDE4 inhibitor in a patient comprising administering to said patient in need thereof a therapeutically effective amount of a compound as claimed in claim 1, or a pharmaceutically acceptable salt thereof.

16. A method for treating an airway disorder in a patient comprising administering to said patient a therapeutically effective amount of a compound as claimed in claim 1, or a pharmaceutically acceptable salt thereof.
